(19) United States
(12) Patent Application Publication
(10) Pub. No.: US 2022/0073641 A1
(43) Pub. Date: Mar. 10, 2022

(54) FUSION PROTEIN OF ANTIBODY THAT RECOGNIZES CANCER CELLS AND MUTANT STREPTAVIDIN

(71) Applicants: The University of Tokyo, Tokyo (JP); SAVID THERAPEUTICS INC., Tokyo (JP)

(72) Inventors: Toshiya TANAKA, Tokyo (JP); Tatsuhiko KODAMA, Tokyo (JP); Takefumi YAMASHITA, Tokyo (JP); Motomu KANAI, Tokyo (JP); Kenzo YAMATSUGU, Tokyo (JP); Toshifumi TATSUMI, Tokyo (JP); Kazuki TAKAHASHI, Tokyo (JP); Shumpei ISHIKAWA, Tokyo (JP); Akira SUGIYAMA, Tokyo (JP); Masanobu TSUKAGOSHI, Tokyo (JP)

(73) Assignees: The University of Tokyo, Tokyo (JP); SAVID THERAPEUTICS INC., Tokyo (JP)

(21) Appl. No.: 17/418,475

(22) PCT Filed: Dec. 27, 2019
(86) PCT No.: PCT/JP2019/051449

§ 371 Date: Jun. 25, 2021

(30) Foreign Application Priority Data
Dec. 28, 2018 (JP) 2018-247363

(51) Int. Cl. C07K 16/30; C07K 14/36; A61K 47/54

(52) U.S. Cl. CPC C07K 16/30; C07K 2319/02; A61K 47/54; C07K 14/36

(57) ABSTRACT

It is an object of the present invention to provide a fusion protein of an antibody that recognizes cancer cells and a mutant streptavidin, which is for use in the treatment or diagnosis of cancer. According to the present invention, provided is a fusion protein having the amino acid sequence as set forth in SEQ ID NO: 2 or SEQ ID NO: 7, a linker sequence, and the amino acid sequence as set forth in SEQ ID NO: 1 (provided that the amino acid sequence portion consisting of 6 histidine residues at the C-terminus thereof may be partially or entirely deleted), from the N-terminal side to the C-terminal side, in this order.

### TECHNICAL FIELD

The present invention relates to a fusion protein of an antibody that recognizes cancer cells and a mutant streptavidin, and the use thereof

### BACKGROUND ART

Avidin and biotin, or streptavidin and biotin have an extremely high affinity between them (Kd=10⁻¹⁵ to 10⁻¹⁴ M). This is one of extremely strong interactions between two biomolecules. At present, the interaction between avidin/streptavidin and biotin has been widely applied in the field of biochemistry, molecular biology, or medicine. A drug delivery method and a retargeting method, in which high binding ability between avidin/streptavidin and biotin is combined with an antibody molecule, have been devised. In connection with these studies, a mutant streptavidin with a reduced affinity for natural biotin and a biotin-modified dimer having a high affinity for the mutant streptavidin with a low affinity for natural biotin are reported in Patent Document 1.

### PRIOR ART DOCUMENTS

### Patent Documents

Patent Document 1: International Publication WO2015/125820

### SUMMARY of Invention

### Object to be Solved by the Invention

It is an object of the present invention to provide a fusion protein of an antibody that recognizes cancer cells and a mutant streptavidin, which is for use in the treatment or diagnosis of cancer. It is another object of the present invention to provide a means for treating cancer or means for diagnosing cancer, in which the above-described fusion protein is used.

### Means for Solving the Object

As a result of intensive studies directed towards achieving the above-described objects, the present inventor has selected an anti-CEA antibody and an anti-HER2 antibody as antibodies that recognize cancer cells, and then, have prepared a fusion protein of the above-described antibody and a mutant streptavidin. Thereafter, the present inventor has found that the proliferation of cancer cells can be suppressed by photoimmunotherapy using the above-described fusion protein and a conjugate of a biotin-modified dimer and a phthalocyanine dye, thereby completing the present invention.

Specifically, according to the present invention, the following inventions are provided.

<1> A fusion protein having the amino acid sequence as set forth in SEQ ID NO:2 or SEQ ID NO: 7, a linker sequence, and the amino acid sequence as set forth in SEQ ID NO: 1 (provided that the amino acid sequence portion consisting of 6 histidine residues at the C-terminus thereof may be partially or entirely deleted), from the N-terminal side to the C-terminal side, in this order.
<2> The fusion protein according to <1>, wherein the number of amino acids of the linker sequence is 5 to 15.
<3> The fusion protein according to <1> or <2>, wherein the linker sequence consists of 4 to 14 glycine residues and 1 cysteine residue.
<4> The fusion protein according to anyone of <1> to <3>, wherein the linker sequence is Gly-Gly-Gly-Gly-Ser-Gly-Gly-Gly-Gly or Gly-Gly-Gly-Gly-Ser-Gly-Gly-Gly-Gly-Gly.
<5> A fusion protein having the amino acid sequence as set forth in SEQ ID NO: 3 or SEQ ID NO: 8 (provided that the amino acid sequence portion consisting of 6 histidine residues at the C-terminus thereof may be partially or entirely deleted).
<6> The fusion protein according to anyone of <1> to <5>, further having a secretory signal sequence.
<7> A fusion protein having the amino acid sequence as set forth in SEQ ID NO: 4 or SEQ ID NO: 9 (provided that the amino acid sequence portion consisting of 6 histidine residues at the C-terminus thereof may be partially or entirely deleted).
<8> A nucleic acid encoding a fusion protein having the amino acid sequence as set forth in SEQ ID NO: 3 or SEQ ID NO: 8 (provided that the amino acid sequence portion consisting of 6 histidine residues at the C-terminus thereof may be partially or entirely deleted).
<9> A nucleic acid encoding a fusion protein having the amino acid sequence as set forth in SEQ ID NO: 4 or SEQ ID NO: 9 (provided that the amino acid sequence portion consisting of 6 histidine residues at the C-terminus thereof may be partially or entirely deleted).
<10> A cancer therapeutic agent or a cancer diagnostic agent, comprising the fusion protein according to any one of <1> to <7>.
<11> A kit for treating or diagnosing cancer, comprising (1) the fusion protein according to anyone of <1> to <7>, and (2) a conjugate of a compound represented by the following formula (1) or a salt thereof and a diagnostic substance or a therapeutic substance:

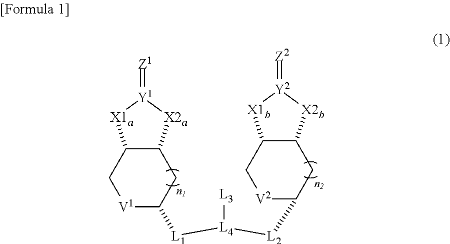

wherein
X1a, X1b, X2a and X2b each independently represent O or NH,
Y¹ and Y² each independently represent C or S,
Z¹ and Z² each independently represent O, S or NH,
V¹ and V² each independently represent S or S*O,
n1 and n2 each independently represent an integer of 0 or 1,
L₁ and L₂ each independently represent a divalent linking group,
L₃ represents a group comprising a functional group capable of binding to the diagnostic substance or the therapeutic substance at the terminus, and
L₄ represents a trivalent linking group.
<12> The kit according to <11>, wherein the diagnostic substance or the therapeutic substance is a phthalocyanine dye.

### Advantageous Effects of Invention

The proliferation of cancer cells can be suppressed by using the fusion protein of the present invention consisting of an antibody that recognizes cancer cells and a mutant streptavidin.

### BRIEF DESCRIPTION OF DRAWINGS

FIG. 1 shows an outline of a domain structure.

FIG. 2 shows a CBB-stained SDS-PAGE electrophoretic pattern of CEA-V2122.

FIG. 3 shows evaluation of the binding performance of CEA-Cupid with an antigen (CEACAM5).

FIG. 4 shows evaluation of the binding performance of CEA-Cupid with a modified biotin.

FIG. 5 shows stained cell images obtained using FITC-labeled CEA-V2122.

FIG. 6 shows timeseries data of stained MKN45 cell images obtained using FITC-labeled CEA-V2122.

FIG. 7 shows photoactivatable compound-binding modified biotins.

FIG. 8 shows in vitro cytotoxicity obtained by a combination of CEA-Cupid and a photoactivatable compound-binding modified biotin.

FIG. 9 shows a change in the size of a tumor mass.

FIG. 10 shows in vivo cytotoxicity obtained by a combination of CEA-Cupid and a photoactivatable compound-binding modified biotin.

FIG. 11 shows histopathological images of in vi cytotoxicity obtained by a combination of CEA-Cupid and a photoactivatable compound-binding modified biotin.

FIG. 12 shows the analysis of pathological sections using an anti-CEACAM5 antibody.

FIG. 13 shows a structural drawing of HER2-V2122.

FIG. 14 shows a CBB-stained SDS-PAGE electrophoretic pattern of HER2-V2122.

FIG. 15 shows evaluation of the binding performance of HER2-Cupid with an antigen (HER2).

FIG. 16 shows evaluation of the binding performance of HER2-Cupid with a modified biotin.

FIG. 17 shows in vitro cytotoxicity obtained by a combination of HER2-Cupid and a photoactivatable compound-binding modified biotin.

### EMBODIMENT OF CARRYING OUT THE INVENTION

Hereinafter, the present invention will be described in more detail.

### <Fusion Protein of Antibody and Mutant Streptavidin>

The fusion protein of the present invention is a fusion protein having the amino acid sequence as set forth in SEQ ID NO: 2 or SEQ ID NO: 7, a linker sequence, and the amino acid sequence as set forth in SEQ ID NO: 1 (provided that the amino acid sequence portion consisting of 6 histidine residues at the C-terminus thereof may be partially or entirely deleted), from the N-terminal side to the C-terminal side, in this order. The fusion protein of the present invention may also be a fusion protein having the amino acid sequence of an anti-desmoglein antibody, a linker sequence, and the amino acid sequence as set forth in SEQ ID NO: 1 (provided that the amino acid sequence portion consisting of 6 histidine residues at the C-terminus thereof may be partially or entirely deleted), from the N-terminal side to the C-terminal side, in this order.

The amino acid sequence as set forth in SEQ ID NO: 2 is the amino acid sequence of an scFv-type anti-CEACAM antibody. The amino acid sequence as set forth in SEQ ID NO: 7 is the amino acid sequence of an scFv-type anti-Her2 antibody.

The amino acid sequence as set forth in SEQ ID NO:1 is the amino acid sequence of a mutant streptavidin, and specifically, this mutant streptavidin is mutant streptavidin LISA314-V2122 described in Example 3 of International Publication WO2015/125820 (SEQ ID NO:4 of International Publication WO2015/125820)(SEQ ID NO: 1 of the description of the present application).

As described above, the fusion protein of the present invention is a fusion protein of an antibody that recognizes cancer cells and a mutant streptavidin.

The linker sequence is not particularly limited, as long as it can achieve the effects of the present invention. The number of amino acids of the linker sequence is preferably 5 to 15, more preferably 7 to 13, and further preferably 9 to 11.

A specific example of the linker sequence may be a sequence consisting of 4 to 14 glycine residues and 1 cysteine residue. As such a linker sequence, an amino acid sequence represented by, for example, (Gly)ₘ-Cys-(Gly)ₙ (wherein m and n each independently represent an integer of 1 to 13) can be used. Specific example of the linker sequence may include Gly-Gly-Gly-Gly-Ser-Gly-Gly-Gly-Gly and Gly-Gly-Gly-Gly-Gly-Ser-Gly-Gly-Gly-Gly-Gly, but the examples are not particularly limited thereto.

A specific example of the fusion protein of the present invention may be a fusion protein having the amino acid sequence as set forth in SEQ ID NO: 3 or SEQ ID NO: 8 (provided that the amino acid sequence portion consisting of 6 histidine residues at the C-terminus thereof may be partially or entirely deleted).

The fusion protein of the present invention may also have a secretory signal sequence at the N-terminus thereof. The fusion protein having a secretory signal sequence may be a fusion protein having the amino acid sequence as set forth in SEQ ID NO: 4 or SEQ ID NO: 9 (provided that the amino acid sequence portion consisting of 6 histidine residues at the C-terminus thereof may be partially or entirely deleted).

According to the present invention, a nucleic acid (for example, DNA) encoding the above-described fusion protein of the present invention is further provided. A specific example of the nucleic acid of the present invention may be a nucleic acid encoding a fusion protein having the amino acid sequence as set forth in SEQ ID NO:3 or SEQ ID NO:8 (provided that the amino acid sequence portion consisting of 6 histidine residues at the C-terminus thereof may be partially or entirely deleted). Another specific example of the nucleic acid of the present invention may be a nucleic acid encoding a fusion protein having the amino acid sequence as set forth in SEQ ID NO: 4 or SEQ ID NO: 9 (provided that the amino acid sequence portion consisting of 6 histidine residues at the C-terminus thereof may be partially or entirely deleted).

A nucleic acid (for example, DNA) encoding the fusion protein of the present invention can be used by being incorporated into a vector. In order to produce the fusion protein of the present invention, a nucleic acid encoding the fusion protein of the present invention is incorporated into an expression vector, and a host is then transformed with this expression vector, so that the fusion protein of the present invention can be expressed in the host.

When Escherichia coli is used as a host, the vector preferably has a replication origin (or) and also has a gene for selecting the transformed host (e.g. a drug-resistance gene that is resistant to drugs, such as ampicillin, tetracycline, kanamycin or chloramphenicol, etc.). Moreover an expression vector preferably has a promoter capable of efficiently expressing the mutant streptavidin of the present invention in a host, such as a lacZ promoter or a T7 promoter. Examples of such a vector include an M13 vector, a pUC vector, pBR322, pBluescript, pCR-Script, pGEX-5X-1 (Pharmacia), “QIAexpress system” (QIAGEN), pEGFP, and pET (in this case, BL21 that expresses T7 RNA polymerase is preferably used as a hot).

A vector can be introduced into a host cell by applying a calcium chloride method or an electroporation method, for example. Further, a sequence that encodes a tag for improving solubility, such as glutathione S-transferase, thioredoxin or a maltose-binding protein, may be added. Still further, a sequence that encodes a tag designed for facilitating purification, such as a polyhistidine tag, a Myc epitope, a hemagglutinin (HA) epitope, a T7 epitope, an Xpress tag, a FLAG tag or other known tag sequences, may also be added.

Other than Escherichia coli, examples of the expression vector include: mammal-derived expression vectors (for example, pcDNA3 (manufactured by Invitrogen), pEGF-BOS (Nucleic Acids. Res. 1990, 18(17), p. 5322), pEF and pCDM8); insect cell-derived expression vectors (for example, “Bac-to-BAC baculovirus expression system” (manufactured by Gibco-BRL) and pBacPAK8); plant-derived expression vectors (for example, pMH1 and pMH2); animal vin-derived expression vectors (for example, pHSV, pMV and pAdexLcw); retrovirus-derived expression vectors (for example, pZIPneo); yeast-derived expression vectors (for example, “Pichia Expression Kit” (manufactured by Invitrogen), pNV11 and SP-Q01); and Bacillus subtilis-derived expression vectors (for example, pPL608 and pKTH50).

When the expression of the present mutant streptavidin in an animal cell such as a CHO cell, a COS cell or an NIH3T3 cell is intended, it is essential for the expression vector to have a promoter necessary for the expression of the mutant streptavidin in such an animal cell, such as an SV40 promoter (Mulligan et al., Nature (1979) 277, 108), an MMLV-LTR promoter, an EF1α promoter (Mizushima et al., Nucleic Acids Res. (1990) 18, 5322) or a CMV promoter. It is more preferable if the expression vector has a gene for selecting the transformation of a cell (for example, a drug-resistance gene capable of determining transformation with the use of drugs (neomycin, G418, etc.)). Examples of a vector having such properties include pMAM, pDR2, pBK-RSV, pBK-CMV, pOPRSV and pOP13.

The type of a host cell, into which the vector is introduced, is not particularly limited. Either prokaryotes or eukaryotes may be used. It is possible to use Escherichia coli or various types of animal cells, for example.

In the case of using a eukaryotic cell, for example, an animal cell, a plant cell or a fungal cell can be used as a host. Examples of an animal cell that can be used herein include: mammalian cells such as a CHO cell, a COS cell, a 3T3 cell, a HeLa cell or a Vero cell; and insect cells such as Sf9, Sf21 or Tn5. When the expression of a large amount of the mutant streptavidin in an animal cell is intended, a CHO cell is particularly preferable. A vector can be introduced into a host cell by a calcium phosphate method, a DEAE-dextran method, a method using cationic ribosome DOTAP (manufactured by Boehringer Mannheim), an electroporation method, a lipofection method or the like.

As a plant cell, a cell from Nicotiana tabacum has been known as a protein-producing system, for example. These cells may be subjected to callus culture. Examples of a known fungal cell include: yeast cells including genus Saccharomyces such as Saccharomyces cerevisiae and filamentous fungi including genus Aspergillus such as Aspergillus niger.

Examples of a procaryotic cell that can be used herein include Escherichia coli (E. coli), such as JM109, DH5α or HB101. Moreover, Bacillus subtilis has been known.

These cells am transformed with the nucleic acid of the present invention, and the transformed cells are then cultured in vitro, so as to obtain the fusion protein of the present invention. The culture can be carried out in accordance with a known culture method. Examples of a culture solution of animal cells that can be used herein include DMEM, MEM, RPMI1640, and IMDM. During the culture, a serum infusion such as fetal calf serum (FCS) may be used in combination, or serum free culture may also be carried out. The pH applied during the culture is preferably approximately pH 6 to 8. The culture is generally carried out at a temperature of approximately 30° C. to 40° C. for approximately 15 to 200 hours. As necessary, medium replacement, ventilation and stirring are carried out. Furthermore, growth factors may also be added to promote the growth of cells.

### <Cancer Therapeutic Agent or Cancer Diagnostic Agent>

The fusion protein of the present invention is useful as a cancer therapeutic agent or a cancer diagnostic agent.

According to the present invention, provided is a kit for treating or diagnosing cancer, comprising (1) the fusion protein of the present invention, and (2) a conjugate of a compound represented by a formula (1) as shown below or a salt thereof, and a diagnostic substance or a therapeutic substance.

By administering the fusion protein of the present invention to a patient, a mutant streptavidin can be accumulated in the body of the patient, specifically into cancer cells. Subsequently, by administering a conjugate of a biotin-modified dimer having an affinity for the mutant streptavidin and a diagnostic substance or a therapeutic substance to the patient, it becomes possible to accumulate the diagnostic substance or the therapeutic substance precisely into the cancer cells.

Otherwise, a complex is prepared by binding the “fusion protein of the present invention” with the “conjugate of a biotin-modified dimer having an affinity for a mutant streptavidin and a diagnostic substance or a therapeutic substance,” and the thus prepared complex can be administered to the patient.

### <Biotin-Modified Dimer>

The biotin-modified dimer is a compound represented by the following formula (1) or a salt thereof and is preferably a compound represented by the following formula (2) or a salt thereof. As such a biotin-modified dimer, the compound described in International Publication WO2015/125820 can be used.

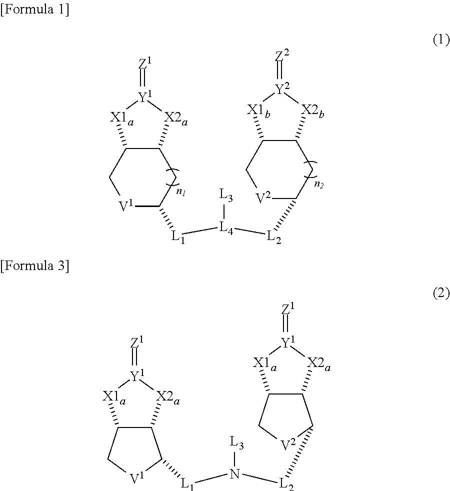

wherein
X1a, X1b, X2a and X2b each independently represent O or NH,
Y¹ and Y² each independently represent C or S,
Z¹ and Z² each independently represent O, S or NH,
V¹ and V² each independently represent S or S⁺—O⁻,
n1 and n2 each independently represent an integer of 0 or 1,
L₁ and L₂ each independently represent a divalent linking group,
L₃ represents a group comprising a functional group capable of binding to the diagnostic substance or
the therapeutic substance (for example, a phthalocyanine dye) at the terminus, and
U represents a trivalent linking group.

In the formula (1) and the formula (2), the portions represented by the following structures:

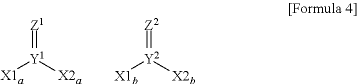

are preferably any one of the following portions, but are not limited thereto:

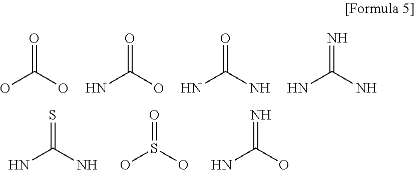

X1a, X1b, X2a and X2b preferably represent NH; Y¹ and Y² preferably represent C; Z¹ and Z² preferably represent NH; and V¹ and V² preferably represent S.

L₁ and L₂ each independently represent a divalent linking group consisting of a combination of groups selected from —CONH—, —NHCO—, —COO—, —OCO—, —CO—, —O—, and an alkylene group containing 1 to 10 carbon atoms.

Preferably, L₁ and L₂ each independently represent a divalent linking group consisting of a combination of groups selected from —CONH—, —NHCO—, —O—, and an alkylene group containing 1 to 10 carbon atoms.

Preferably, L₁ and L₂ each independently represent a divalent linking group consisting of a combination of groups selected from —CONH—, —NHCO—, and an alkylene group containing 1 to 10 carbon atoms

L₄ represents a trivalent linking group, and is preferably the following:

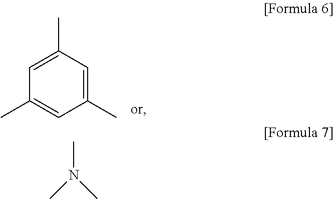

(which is a benzene-derived trivalent linking group or a nitrogen atom).

La is preferably a group consisting of a combination of groups selected from —CONH—, —NHCO—, —COO—, —OCO—, —CO—, —O—, and an alkylene group containing 1 to 10 carbon atoms, and further comprising an amino group at the terminus.

### <Conjugate of Biotin-Modified Dimer and Diagnostic Substance or Therapeutic Substance>

By binding a diagnostic substance or a therapeutic substance to a biotin-modified dimer, a conjugate of the biotin-modified dimer and the diagnostic substance or the therapeutic substance can be prepared. Examples of the diagnostic substance or the therapeutic substance may include a fluorochrome, a chemiluminescent agent, a radioisotope, a sensitizer consisting of a metal compound or the like, a neutron-capturing agent consisting of a metal compound or the like, a phthalocyanine dye, a low-molecular-weight compound, micro- or nano-bubbles, and a protein. Preferably, a phthalocyanine dye can be used.

### <Phthalocyanine Dye>

The phthalocyanine dye is preferably a silicon phthalocyanine dye. Specific examples of the phthalocyanine dye, such as IRDye (registered trademark) 700DX, are described in, for example, U.S. Pat. No. 7,005,518.

As such a phthalocyanine dye, a dye represented by the following formula (21) can be used. As an example, a dye represented by the following formula (22) can be used.

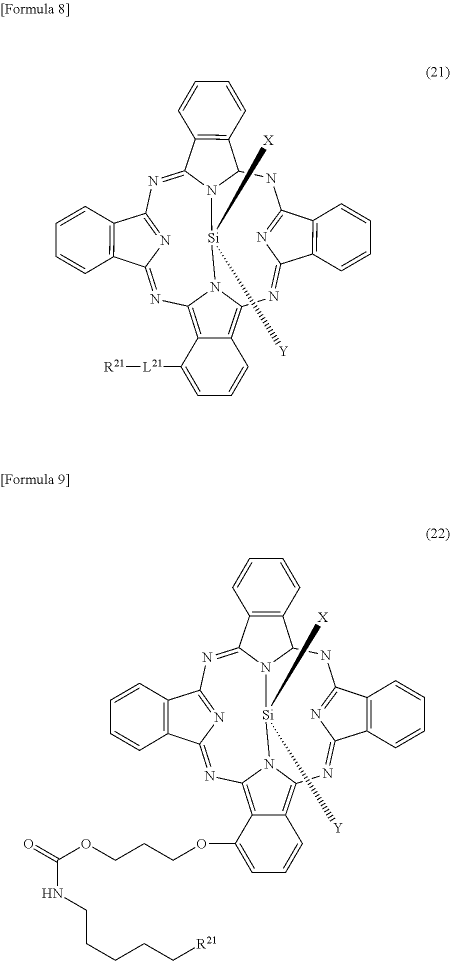

In the above formulae, L²¹ represents a divalent linking group, R²¹ represents a functional group capable of binding to the compound represented by the formula (1) or a salt thereof.

In the above formulae, X and Y each independently represent a hydrophilic group, —OH, a hydrogen atom, or a substituent Examples of the substituent used herein may include, but are not particularly limited to, a halogen atom (a fluorine atom), a substituent containing a carbon atom (a hydrocarbon group, etc.), and a substituent containing a nitrogen atom (an amino group, etc.).

Specific examples of X and Y may include:

(i) a case where both X and Y ae hydrophilic groups;
(ii) a case where either X or Y is a hydrophilic group, and the other is —OH or a hydrogen atom; and
(iii) a case where X and Y are —OH or hydrogen atoms.

The hydrophilic group(s) represented by X and/or Y are not particularly limited. One example is shown below.

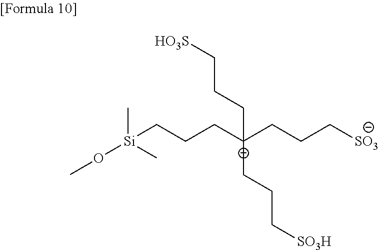

As a phthalocyanine dye, a commercially available product such as IRDye (registered trademark) 700DX can be used. In the present invention, an NHS ester of IRDye (registered trademark) 700DX is used, and is allowed to react with a biotin-modified dimer having an amino group to produce a conjugate. Other variations of IRDye (registered trademark) 700DX are described in U.S. Pat. No. 7,005,518, and those can also be used.

R²¹ represents a functional group capable of binding to the compound represented by the formula (1) or a salt thereof. R²¹ is preferably a functional group that can react with a carboxyl group, amine or a thiol group on the biotin-modified dimer and can bind thereto. Preferred examples of R²¹ may include, but are not particularly limited to, activated ester, halogenated acyl, halogenated alkyl, appropriately substituted amine, anhydride, caboxylic acid, carbodiimide, hydroxyl, iodoacetamide, isocyanate, isothiocyanate, maleimide, NHS ester, phosphoramidite, sulfonic acid ester, thiol, and thiocyanate.

L²¹ represents a divalent linking group, and for example, it may include: any given combination of an ether, thioether, amine, ester, carbamate, urea, thiourea, oxy or amide bond, or a single, double, triple or aromatic carbon-carbon bond; or a phosphorus-oxygen, phosphorus-sulfur, nitrogen-nitrogen, nitrogen-oxygen, or nitrogen-platinum bond; or an aromatic or heteroaromatic bond. L²¹ is preferably a group consisting of a combination of groups selected from —CONH—, —NHCO—, —COO—, —OCO—, —CO—, —O—, and an alkylene group containing 1 to 10 carbon atoms.

-L²¹-R²¹ may include a phosphoramidite group, NHS ester, active carboxylic acid, thiocyanate, isothiocyanate, maleimide, and iodoacetamide.

L²¹ represents a —(CH₂)ₙ— group, and in the formula, n is an integer of 1 to 10, and is preferably an integer of 1 to 4. As one example, -L²¹-R²¹ is —O—(CH₂)₃—OC(O)—NH—(CH₂)₅—C(O)O—N-succinimidyl.

### <Photoimmunotherapy>

Photoimmunotherapy is a therapeutic method of using a photosensitizer and an irradiation light to destroy specific cells in a body. When a photosensitizer is exposed to a light with a specific wavelength, it generates cytotoxic reactive oxygen species capable of inducing apoptosis, necrosis, and/or autophagy to around cells. For example, Japanese Patent No. 6127045 discloses a method of killing cells, comprising: a step of allowing cells comprising a cell surface protein to come into contact with a therapeutically effective amount of one or more antibodies-R700 molecules, wherein the antibodies specifically bind to the cell surface protein; a step of irradiating the cells with a light at a wavelength of 660 to 740 nm and at a dose of at least 1 Jcm⁻²; and a step of allowing the cells to come into contact with one or more therapeutic agents at approximately 0 to 8 hours after the irradiation, thereby killing the cells. JP Patent Publication (Kohyo) No. 2017-524659 A discloses a method of inducing cytotoxicity to a subject affected with a disease or a pathology, comprising: (a) administering to a subject, a therapeutically effective drug comprising a phthalocyanine dye such as IRDye (registered trademark) 700DX conjugated with a probe specifically binding to the cell of the subject; and (b) irradiating the cell with an appropriate excitation light in an amount effective for inducing cell death.

The fusion protein of the present invention and the conjugate of a biotin-modified dimer and a phthalocyanine dye are administered to a subject, and the cells we then irradiated with an excitation light in an amount effective for suppression of cell proliferation or induction of cell death, so that the cell proliferation can be suppressed or the cell death can be induced, and thereby the subject can be treated.

Preferably, the fusion protein of the present invention and the conjugate of a biotin-modified dimer and a phthalocyanine dye are administered to a subject, and the cells are then irradiated with an excitation light in an amount effective for suppression of cell proliferation or induction of cell death, so that the cell proliferation can be suppressed or the cell death can be induced, and thereby the subject can be treated.

The subject used herein includes humans and non-human animals. Examples of the subject may include humans and experimental animals such as mice. The subject is preferably affected with a disease regarding which suppression of cell proliferation or induction of cell death is desired. For example, the subject is affected with cancer or solid tumor.

Examples of the “cancer” may include carcinoma, lymphoma, blastoma, sarcoma, and leukemia or malignant lymphoma. Specific examples of the cancer may include squamous cell carcinoma (e.g., epithelial squamous cell carcinoma), lung cancer including small cell lung cancer, non-small cell lung cancer (“NSCLC”), pulmonary adenocarcinoma and pulmonary squamous cell carcinoma, peritoneal cancer, hepatocarcinoma, corpus ventriculi or stomach cancer, including digestive cancer, pancreatic cancer, glioblastoma, cervical cancer, ovarian cancer, liver cancer, bladder caner, hepatocellular cancer, breast cancer, colon cancer, rectal caner, colorectal cancer, endometrial membrane cancer or endometrial carcinoma, salivary gland carcinoma, kidney or renal region cancer, prostate cancer, vulvar cancer, thyroid cancer, hepatocellular carcinoma, anal carcinoma, penile carcinoma, and head and neck cancer.

The solid tumor means a benign or malignant, abnormal cell mass that generally does not contain a capsule. Examples of the solid tumor may include glioma, astrocytoma, medulloblastoma, craniopharyngioma, ependymoma, pineal gland tumor, hemangioblastoma acoustic neuroma, oligodendrocyte, meningioma, melanoma, neuroblastoma, and retinoblastoma.

Examples of the administration method to the subject may include, but are not limited to, a local route, an injection (a subcutaneous injection, an intramuscular injection, an intradermal injection, an intraperitoneal injection, an intratumoral injection, an intravenous injection, etc.), an oral route, an ocular route, a sublingual route, a rectal route, a percutaneous route, an intranasal route, a vaginal route, and an inhalation route.

It is preferable that the conjugate of a biotin-modified dimer and a phthalocyanine dye and the fusion protein of the present invention are each administered in a therapeutically effective amount. Regarding each of the above-described conjugate and fusion protein, the therapeutically effective amount per 60 kg is at least 05 mg (mg/60 kg), at least 5 mg/60 kg, at least 10 mg/60 kg, at least 20 mg/60 kg, at least 30 mg/60 kg, or at least 50 mg/60 kg. For example, when it is intravenously administered, the applied dose is 1 mg/60 kg, 2 mg/60 kg, 5 mg/60 kg, 20 mg/60 kg, or 50 mg/60 kg, and it is, for example, 0.5 to 50 mg/60 kg. In another example, the therapeutically effective amount is at least 100 μg/kg, at least 500 μg/kg or at least 500 μg/kg, and it is, for example, at least 10 μg/kg. For example, when it is intratumorally or intraperitonealy administered, the dose is 100 μg/kg, 250 μg/kg, approximately 500 μg/kg, 750 μg/kg, or 1000 μg/kg, and it is, for example, 10 μg/kg to 1000 μg/kg. In one example, when it is administered in the form of a solution for local administration, the therapeutically effective amount is 10 μg/ml, 20 μg/ml, 30 μg/ml, 40 μg/ml, 50 μg/ml, 60 μg/ml, 70 μg/ml, 80 μg/ml, 90 μg/m, 100 μg/ml or the like, or it is 20 μg/ml to 100 μg/ml, or it is at least 500 μg/ml, or at least 1 μg/ml.

The above-described dose can be administered once or divided doses over several administrations (2, 3, or 4 times, etc.), or as a single preparation.

The conjugate of a biotin-modified dimer and a phthalocyanine dye and the fusion protein of the present invention can be each administered alone, or can also be administered in the presence of a pharmaceutically acceptable carrier, or can also be administered in the presence of other therapeutic agents (other anticancer agents, etc.).

The conjugate of a biotin-modified dimer and a phthalocyanine dye and the fusion protein of the present invention can bind to target cells or target tissues, such as circulating tumor cells or solid tumor cells. Thereafter, the target cells or tissues are irradiated with a light, so that the above-described conjugate or complex can absorb the light and can damage or destroy the target cells or tissues.

In the photoimmunotherapy, the wavelength of the irradiation light is preferably 660 to 740 nm, and the irradiation light has a wavelength of for example, 660 nm, 670 nm, 680 nm, 690 nm, 700 nm, 710 nm, 720 nm, 730 nm, or 740 nm. Light irradiation may be carried out using a device equipped with a near infrared (NIR) light emitting diode.

The light irradiation amount is at least 1 J/cm², for example, at least 4 J/cm², at least 10 J/cm², at least 15 J/cm², at least 20 J/cm², at least 50 J/cm², or at least 100 J/cm². It is, for example, 1 to 500 J/cm². Light irradiation may be carried out several times (e.g., 2, 3, 4, 5, 6, 7, 8, 9, or 10 times).

The present invention will be more specifically described in the following examples. However, these examples are not intended to limit the scope of the present invention.

### EXAMPLES

### Production Example 1

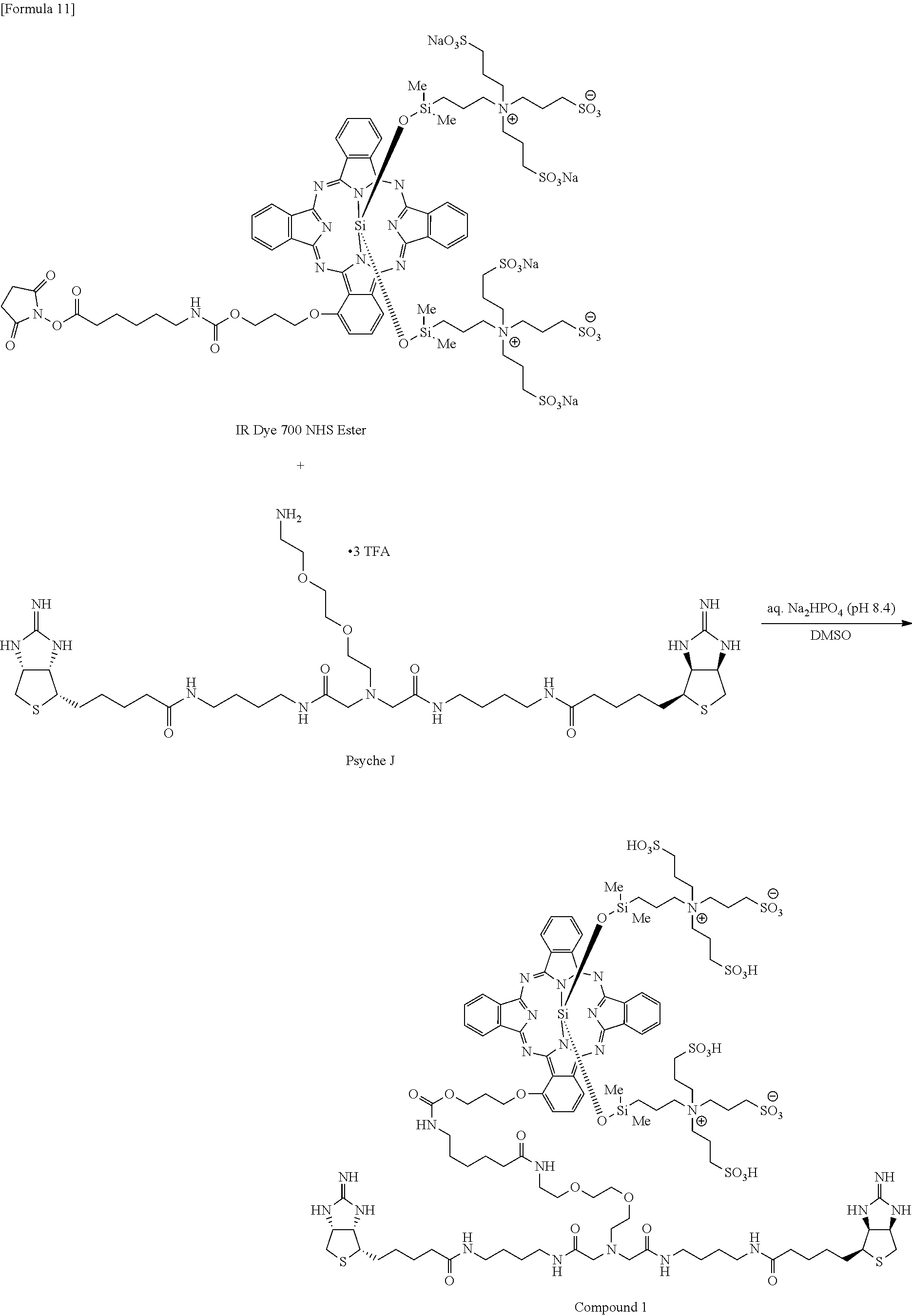

IR Dye 700 NHS Ester (3.0 mg, 1.5 μmol), a disodium hydrogen phosphate buffer (pH 8.4, 144 μL) and dimethyl sulfoxide (120 μL) were added to Psyche J (1.8 mg, 1.6 μmol), light was then shielded with an aluminum foil, and the mixture was then stirred at room temperature for 24 hours. Thereafter, the reaction solution diluted with water to 500 μL was purified by reverse phase HPLC (gradient: 0% for 5 min; 0-100% for 100 min acetonitrile in a 50 mM triethylammonium acetate aqueous solution (pH 6.5), retention time=47.2 min, YMC-Triart C18, flow rate=3.5 mL/min) to obtain a target compound 1 (dark blue green). Quantification was carried out from the reported molar absorption coefficient of IR700 at 694 nm in water (165,000 (M⁻¹ cm⁻¹)), and the yield (1.0 μmol, 72%) was calculated.

LRMS (ESI): m/z 1303 [M+2H]²⁺, 869 [M+3H]³⁺

### Production Example 2

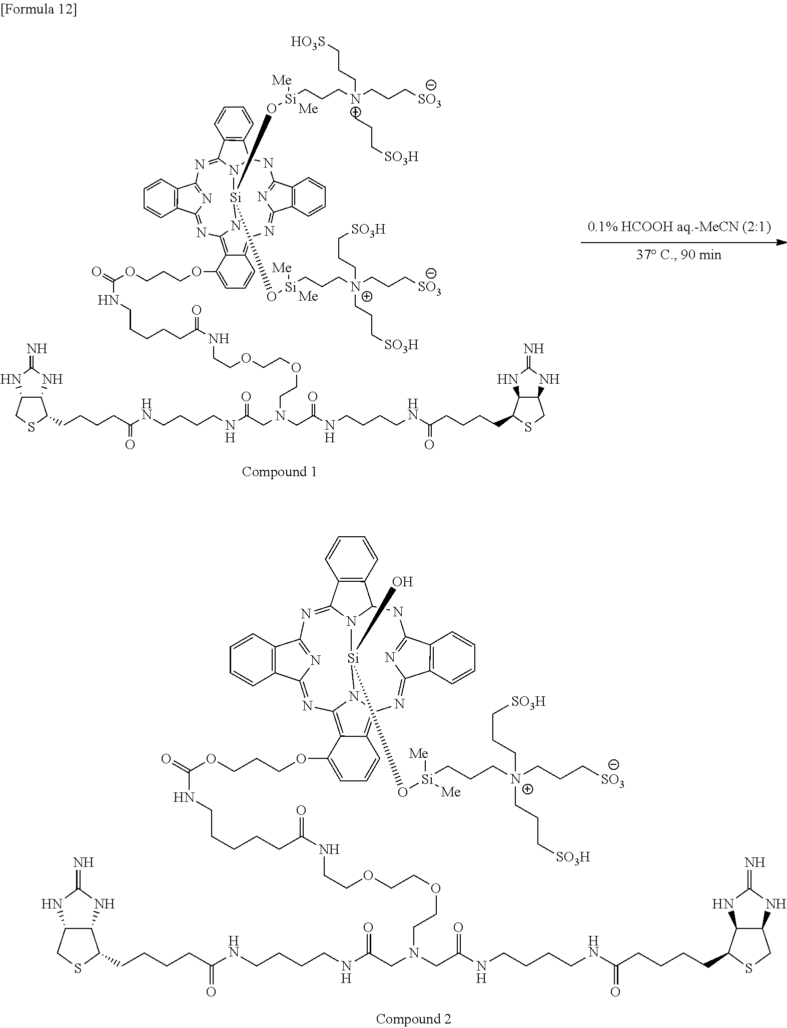

A 0.1% formic acid aqueous solution (266 μL) and acetonitrile (133 μL) were added to the compound 1 (0375 mg, 144 nmol), and the obtained mixture was stirred with a Vortex mixer and was then centrifuged. The resultant was left at rest under conditions of in a darkroom at 37° C. for 90 minutes. The resulting solution was purified by reverse phase HPLC (gradient: 0% for 5 in 0-100% for 100 min acetonitrile in a 50 mM triethylammonium acetate aqueous solution (pH 6.5), retention time=51.6 min, YMC-Triart C18, flow rate=3.5 mL/min) to obtain a target compound 2 (dark blue grew). Quantification was carried out in the same manner as described above, and the yield (48 nmol, 33%) was calculated.

LRMS (ESI): m/z 1054 [M-H₂O-+2H]²⁺, 703 [M-H₂O+3H]³⁺

### Production Example 3

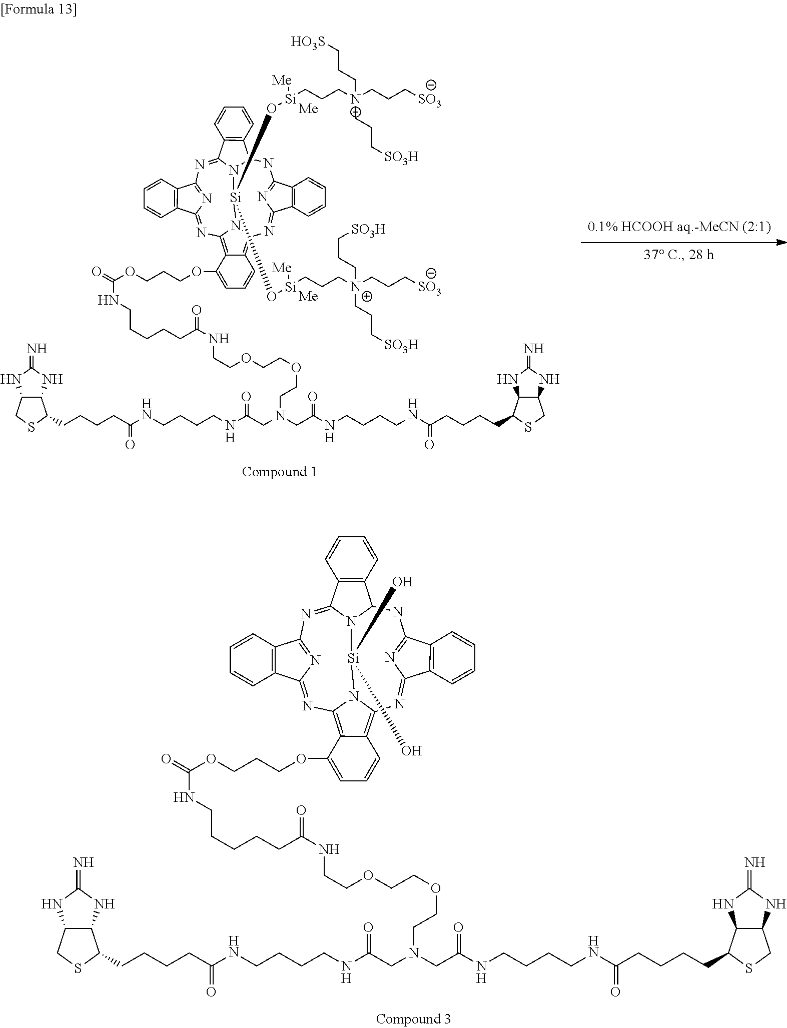

A 0.1% formic acid aqueous solution (266 μL) and acetonitrile (133 μL) were added to the compound 1 (0.375 mg, 144 nmol), and the obtained mixture was stirred with a Vortex mixer and was then centrifuged. The resultant was left at rest under conditions of in a darkroom at 37° C. for 28 hours. The resulting solution was purified by reverse phase HPLC (gradient 0% for 5 min 0-100% for 100 min acetonitrile in a 50 mM triethylammonium acetate aqueous solution (pH 6.5), retention time=56.2 min, YMC-Triart C18, flow rate=3.5 mL/min) to obtain a target compound 3 (dark blue green). Quantification was carried out in the same manner as described above, and the yield (15 nmol, 10%) was calculated.

LRMS (ESI): m/z 813 [M-H₂O—2H]²⁺, 542 [M-H₂O+3H]³⁺

### Example 1: Expression and Purification of CEA-V2122 Protein

V2122 is the mutant streptavidin described in Example 3 of International Publication WO2015/125820 (SEQ ID NO:4 shown in International Publication WO2015/125820). The amino acid sequence of V2122 (a sequence having a 6×His tag at the C-terminus) is as set forth in SEQ ID NO: 1 in the sequence listing.

scFv-V2122 is prepared by binding a single-chain antibody (scFv) against CEACAM5 with the above-described V2122. This scFv-type anti-CEACAM5 antibody is an scFv sequence described in a patent document U.S. Pat. No. 7,626,011B2. The amino acid sequence of the scFv-type anti-CEACAM5 antibody is as set forth in SEQ ID NO:2 in the sequence listing. In addition, the amino acid sequence of CEA-V2122 prepared by binding the scFv-type anti-CEACAM5 antibody with V2122 via an amino acid linker (GGGGSGGGG) (SEQ ID NO: 11) is as set forth in SEQ ID NO: 3 in the sequence listing.

For the expression of a CEA-V2122 fusion protein, the DNA codon of a CEA-V2122 gene sequence, in which a pelB signal for secretion and expression in Escherichia coli had been incorporated into the N-terminus and a 6×His-Tag sequence had been incorporated into the C-terminus, was optimized for Escherichia coli, thereby synthetizing an artificial gene. This amino acid sequence is as se forth in SEQ ID NO: 4 in the sequence listing, and the DNA sequence is as set forth in SEQ ID NO:5 in the sequence listing. Moreover, an outline of a domain structure is shown in FIG. 1.

As a specific protein expression vector, a vector prepared by incorporating a chaperone skp gene into MCS2 of a pETDuet1 vector was used. Regarding the skp gene, the DNA codon was optimized for Escherichia coli based on the amino acid sequence as set forth in SEQ ID NO: 6 in the sequence listing, thereby synthesizing an artificial gene. The synthesized skp gene was amplified by PCR, using the primers (AAGGAGATATACATATGGATAAAATTGCCATTGTTAATAT (SEQ ID NO: 12), and TTGAGATCTOCCATATGTTATTTCACTTTCAGAACG (SEQ ID NO: 13)), and the amplified gene was then cloned into MCS2 of the pETDuel vector linearized with the restriction enzyme NdeI, using In-Fusion HD Cloning Kit, so as to obtain pETDuet_skp. Subsequently, the CEA-V2122 gene was incorporated into MCS1 of pETDuet_skp. Specifically the artificially synthesized CEA-V2122 gene was amplified by PCR, using the primers (AGAAGGAGATATACCATGAAATATCTGCTGCCGAC (SEQ ID NO: 14), and CGCCGAGCTCGAATTTTAATGATGGTGATGATGATG (SEQ ID NO: 15)). Moreover, pETDuet_skp was linearized by PCR, using the primers (GGTATATCCCTTCTTAAAGTTAAAC (SEQ ID NO: 16), and AATTCGAGCTCGGCGCGCCTGCAG (SEQ ID NO: 17)). The CEA-V2122 amplified by PCR and the linearized pETDuet_skp were subjected to cloning using In-Fusion HD Cloning Kit. The cloned vector was confirmed by sequencing, in terms of the gene sequence incorporated therein, and thereafter, it was referred to as pETDuet_CEA-V2122_skp.

For the expression of the protein, pETDuet_CEA-V2122_skp was transformed into BL21(DE3) (Nippon Gene Co., Ltd.), which was then pre-cultured in 2×YT medium (SIGMA-ALDRICH) at 37° C. overnight. The medium used in the pre-culture was added to anew medium for 100-fold dilution, and culture was then carried out at 37° C., until OD (600 nm) became 0.5 to 2.0. Subsequently, IPTG was added to the culture to a final concentration of 0.5 mM, and the obtained mixture was then cultured at 37° C. for 4 hours. Thereafter, a culture supernatant was recovered and was then preserved at 4° C.

The CEA-V2122 protein was roughly purified according to a batch method utilizing 6×His-Tag added to the C-terminus. Specifically, cOmplete His-Tag Purification Resin equilibrated with buffer A (50 mM Tis-HCl, 0.2 M NaCl, 1 mM EDTA, and 5 mM Imidazole; pH 8.0) was added to the culture supernatant preserved at 4° C. The obtained mixture was stirred for 2 hours to overnight at 4° C., so that the protein was allowed to bind to the resin. Subsequently, the resin was recovered into a column, and a 20 column volume of washing operation was performed with buffer A. Thereafter a roughly purified product of CEA-V2122 was recovered by elution with buffer B (50 mM Tis-HCl, 0.2 M NaCl, 1 mM EDTA, and 400 mM Imidazole; pH 8.0).

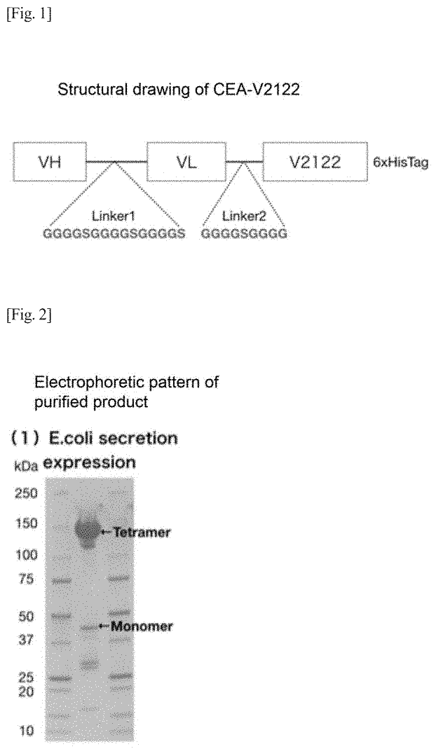

Subsequently, the roughly purified product was purified using a Protein L column. Specifically, 1 mL of Capto L (GE Healthcare Life Sciences) was filled into a PD-10 column, and was then equilibrated with 10 column volume of PBS, and the aforementioned roughly purified product was then applied then to. Thereafter the resultant was washed with 10 column volume of PBS, was then eluted with 10 mM glycine hydrochloride (pH 2.0), and was then subjected to centrifugal concentration using Vivaspin Turbo 15 (MWCO 100,000). Moreover, using PD-10 (GE Healthcare Life Science), the buffer was replaced with PBS, and centrifugal concentration was further carried out using Vivaspin Turbo 4 (MWCO 100,000) to obtain a finally purified product. After completion of SDS-PAGE electrophoresis, the purity of tetramer CEA-V2122 was assayed by CBB staining. The results are shown in FIG. 2. As an SDS-PAGE gel, Mini-PROTEAN TGX 4-15% (Bio-Rad) was used, and as a CBB staining solution, Bullet CBB Stain One (Ready To Use) (Nacalai Tesque, Inc.) was used.

From FIG. 2, it was confirmed that the purified CEA-V2122 comprises an approximately 150 kDa tetramer as a main component.

### Example 2: Evaluation of Performance of CEA-V2122 by SPR

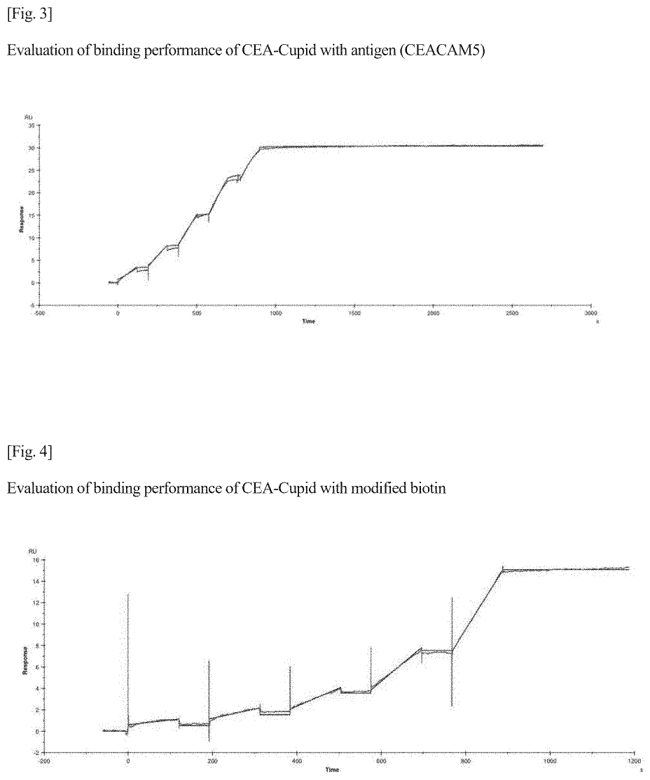

The affinity of CEA-V2122 for the antigen CEACAM5 was evaluated using a surface plasmon resonance (SPR) measuring device, Biacore T200 (GE Healthcare Life Sciences). Specifically, Recombinant Human CEACAM-5/CD66e Protein, CF (R & D SYSTEMS) was immobilized on Sensor Chip CM5 (GE Healthcare Life Sciences) using an amine-coupling kit (GE Healthcare Life Sciences). The final amount of the ligand immobilized was 279 RU. Moreover, with regard to the purified CEA-V2122, two-fold serial dilutions from 1E-08 M to 6.25E-10 M were prepared as analytes. Regarding interaction analysis, data were obtained by single-cycle kinetics analysis. Using Biacore T200 Evaluation Software, version 2.0, the obtained data were subjected to curve fitting in a bivalent analysis mode, and the following values were obtained: ka1=3.208E+5, and kd1=3.461E−7. Moreover, since evaluation can be carried out at K_{D}=kd1/ka1 in the bivalent analysis, the evaluation value K_{D}=kd1/ka1=3.461E−7/3.208E+5=1.078E−12 was obtained. These results are shown in FIG. 3.

From the K_{D} value in the sensorgram shown in FIG. 3, it was confirmed that CEA-V2122 strongly binds to CEACAM5.

Furthermore, the interaction between CEA-V2122 and a modified biotin was also analyzed using Biacore T200. The modified biotin was specifically the title compound 14 described in Example 1 of International Publication WO2018/07239. In addition, the analysis method was specifically as follows. That is, an amine-coupling kit was used, a target value was set to be 5000 RU, and the purified CEA-V2122 was immobilized on Sensor Chip CM5. With regard to the concentrations of the analytes, 5 types of two-fold serial dilutions from 1E-08 M to 6.25E-10 M were used. Regarding interaction analysis, data were obtained by single-cycle kinetics analysis. Using Biacore T200 Evaluation Software, version 2.0, the obtained data were subjected to curve fitting in a bivalent analysis mode, and the following values were obtained: ka1=3.792E+4, and kd1=4.424E−6. Moreover, since evaluation can be carried out at K_{D}=kd1/ka1 in the bivalent analysis, the evaluation value K_{D}=kd1/ka1=3.792E+4/4.424E−6=1.167E−10 was obtained. These results are shown in FIG. 4.

From the K_{D} value in the sensorgram shown in FIG. 4, it was confined that CEA-V2122 strongly binds to a modified biotin.

### Example 3: Cell Staining of CEACAM5-Expressing Cell Line Using FITC-Labeled CEA-V2122

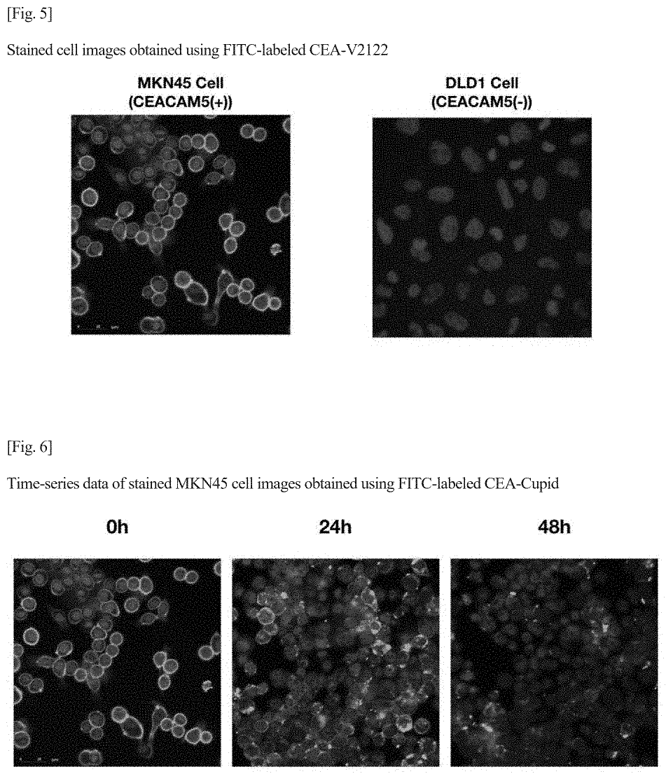

In order to stain a CEACAM5 expression-positive cancer cell line, FTC labeling was carried out on the cell line, using 100 μg of a purified CEA-V2122 protein. Specifically, labeling was carried out using Fluorescein Labeling Kit—NH₂ (DOJINDO LABORATORIES) in accordance with the dosage and administration described in the operating manual included with the kit, and the obtained product was defined to be CEA-V2122-FITC. Specific staining of the CEACAM5 expression-positive cancer cell line is as follows. That is, CEACAM5-positive human stomach cancer-derived MKN-45 cells and CEACAM5-negative human colon cancer-derived DLD1 cells were each seeded on a CELLSTAR, μClear, 96-well plate (Greiner) to a cell density of 2.0×10⁴ cells/well, and thereafter, the cells were cultured overnight. Subsequently, a culture solution containing 20 nM CEA-V2122-FITC and 1 μM Hoechist was added to the 96-well plate to a concentration of 100 μL/well, and the obtained mixture was then reacted at 4° C. for 30 minutes. Thereafter, each image was taken using In Cell Analyzer 6000 (GE Healthcare Life Sciences). The results are shown in FIG. 5 and FIG. 6.

From the results shown in FIG. 5, it was confirmed that CEA-V2122-FITC specifically recognizes CEACAM5 on the surface of the cell membrane. In addition, from the results shown in FIG. 6, it was confirmed that after CEA-V2122-FITC has bound to CEACAM5, the CEACAM5 stays on the surface of the cell membrane.

### Example 4: In Vivo Cytotoxicity Test Using CEA-V2122 and Photoactivatable Compound-Labeled Modified Biotins

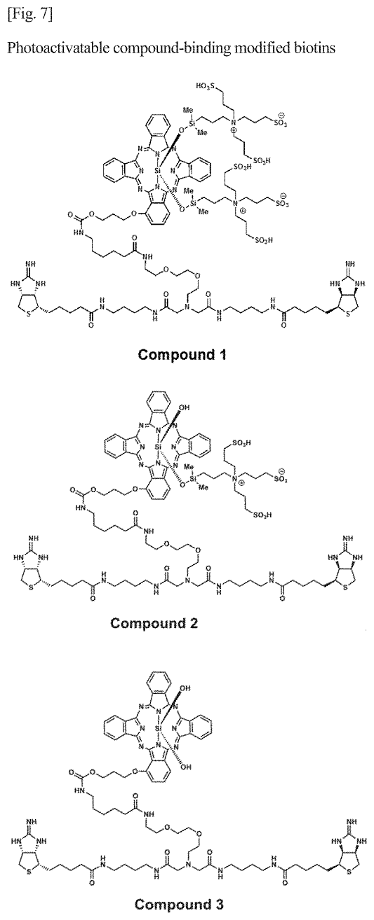

A cytotoxicity test was carried out using the photoactivatable compound-labeled modified biotins, namely, Compound 1, Compound 2, and Compound 3. These compounds are described in Japanese Patent Application No. 2018-149295. Compound 1, Compound 2, and Compound 3 are shown in FIG. 7. Specifically, MKN45 cells were seeded on a 96-well plate for cell culture, so that the cell count became 5×10³ cells/well and the amount of the culture solution became 50 μL/well, and thereafter, the cells were cultured overnight. A solution containing a complex of CEA-V2122 and a photoactivatable compound-labeled modified biotin was prepared, so that the molar ratio of CEA-V2122 and each compound became 1:2, and the solution was then incubated at mom temperature for 10 minutes. Thereafter, the concentration of the reaction solution was adjusted with a culture solution, so that the final concentration of CEA-V2122 became 10 μg/mL. Regarding serial dilutions, 20 μg/mL was set to be an initial concentration, from 4-fold serial dilutions (5.0 μg/mL, 1.25 μg/mL, 0.312 μg/mL, and 0.078 μg/mL), 5 complex serial dilution solutions were prepared. Besides, a medium alone that contained no complex was used as a zero control.

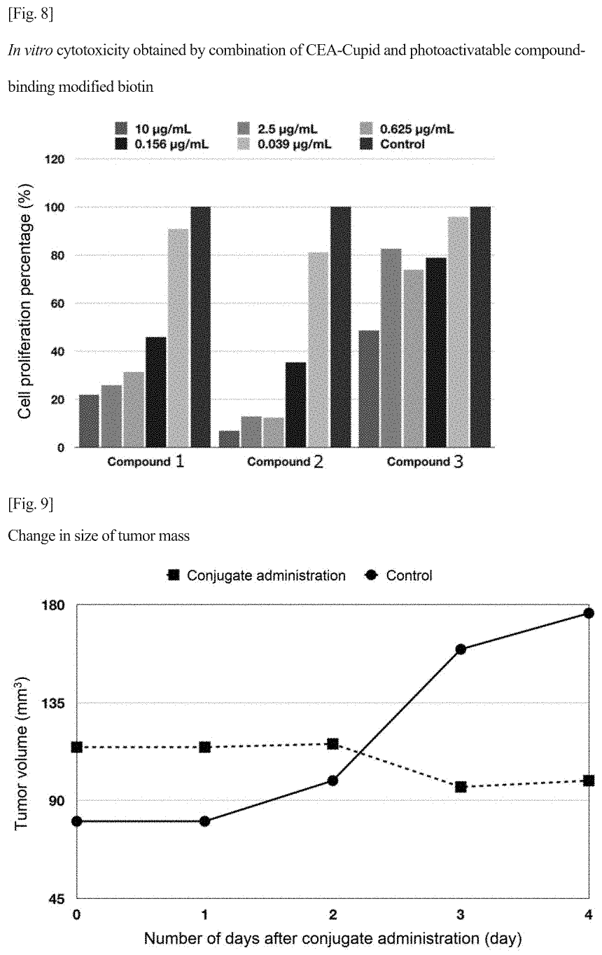

Such complex serial dilution solutions were each added in an amount of 50 μL/well to the cells cultured overnight, so that the final concentrations became 10 μg/mL, 2.5 μg/mL, 0.625 μg/mL, 0.156 μg/mL, and 0.039 μg/mL One hour and two hours after addition of the complex, the cells were irradiated with a light, using LED emitting a light having a wavelength of 690±10 nm, so that the irradiation energy became 100 J/cm². Thereafter, the cells were cultured for 48 hours, and thereafter, a comparison was made in teams of the number of surviving cells, using Cell Counting Kit-8 (DOJINDO LABORATORIES). Each condition was set to be n=3. The dosage and administration were determined in accordance with the instruction manuals included with the kit, and after addition of the reagent, the mixture was incubated for 1.5 hours at 37° C., in a CO₂ incubator. Thereafter, the absorbance at 450 nm was measured, and the mean value was then calculated, followed by background collection. The control was set to be 100%, and the ratio of cell proliferation to the control under each condition was calculated. The results are shown in FIG. 8.

As shown in FIG. 8, it was confirmed that the complex of CEA-V2122, and Compound 1, Compound 2 or Compound 3 exhibits cytotoxicity in a concentration-dependent manner.

### Example 5: In Vivo Cytotoxicity Test Using CEA-V2122 and Photoactivatable Compound-Labeled Modified Biotin, in which Xenograft Mouse Models are Employed

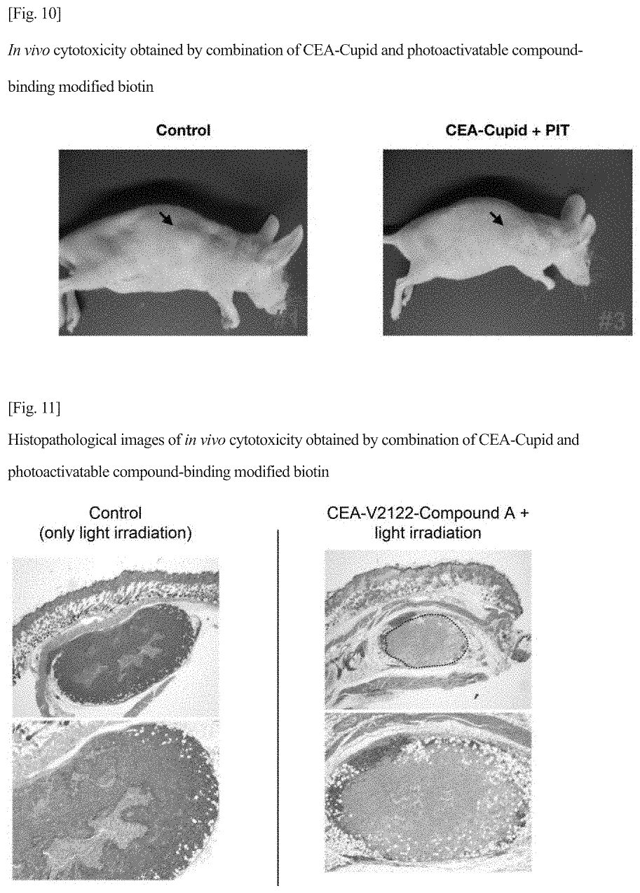

MKN45 cells were subcutaneously transplanted into nude mice to produce xenograft mouse models. That is, 4-week-old female nude mice were purchased, and the mice were then acclimated for 1 week. Thereafter, the MKN45 cells were transplanted in a cell count of 2×10⁵ cells per mouse into the subcutis thereof. Approximately 10 days after completion of the transplantation, 100 μg of a complex of CEA-V2122 and a photoactivatable compound-labeled modified biotin (Compound 1), as described in Experiment Example 4, was administered to the mice via the caudal vein thereof. Using an LED light source emitting a light having a wavelength of 690 nm (USHIO OPTO SEMICONDUCTORS, INC., LD690D-66-60-550.), T-Cube LED Driver (THORLABS, NC0713145), and T-CUBE 15V POWER SUPPLY (THORLABS, TPS001), 6 hours after the administration, the mice were irradiated with the light having a wavelength of 690 nm, so that the irradiation energy became 230 J/cm². Twenty-four hours after administration of the complex, the mice were irradiated again with the light having a wavelength of 690 nm, so that the irradiation energy became 230 J/cm². A graph showing a change in the tumor volume is shown in FIG. 9. Photographs of the mice 5 days after administration of the complex we shown in FIG. 10. Individual mice were euthanized on the 7th day after administration of the complex and were then excised, and the tumor portions were subjected to pathological analysis. The specific method is as follows. That is, the subcutaneous tumor of each mouse and the peripheral tissues thereof were excised as a mass, and the mass was then immersed in a 4% paraformaldehyde solution (Wako Pure Chemical Industries, Ltd., 163-20145) at mom temperature overnight, so that the tissues were fixed. The thus fixed subcutaneous tumor tissues were divided to a thickness of approximately 3 to 5 mm, so as to produce paraffin-embedded blocks. After that, using a microtome, sliced pathological specimens each having a thickness of 4 μm were produced.

Regarding hematoxylin and eosin staining, the sliced pathological specimen was immersed in a xylene solution (Wako Pure Chemical Industries, Ltd., 241-00091) at room temperature for 10 minutes to perform deparaffinization and thereafter hematoxylin staining (Sakura Finetek Japan Co., Ltd., #8650) and eosin staining (Sakura Finetek Japan Co., Ltd., #8660) were carried out. The results are shown in FIG. 11.

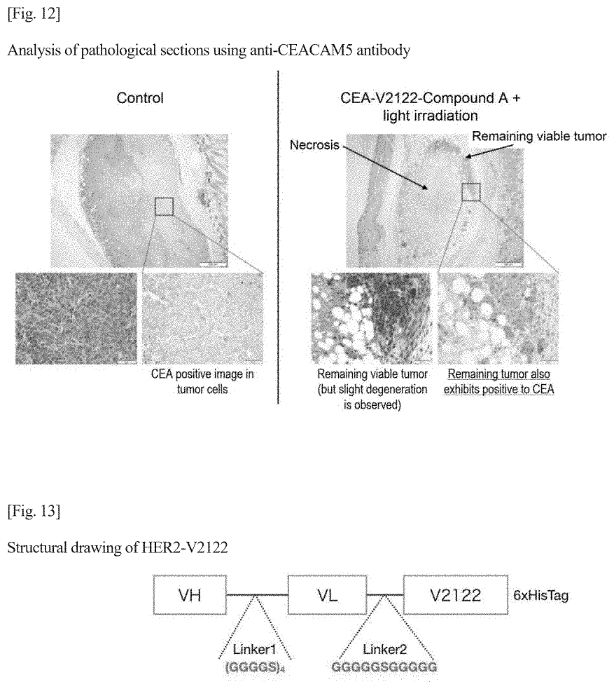

Moreover immunostaining on CEACAM5 was carried out as follows. The sliced pathological specimen was immersed in a xylene solution (Wako Pure Chemical Industries, Ltd., 241-00091) at room temperature for 10 minutes to perform deparaffinization, and thereafter, the antigen was activated by performing an autoclave treatment (121° C., 5 minutes) using a citrate buffer (pH=6.0). Thereafter the resulting specimen was immersed in a 0.3% hydrogen peroxide (Wako Pure Chemical Industries, Ltd., 08104215)/methanol (Wako Pure Chemical Industries, Ltd., 137-01823) solution at room temperature for 10 minutes to remove endogenous peroxidase. Non-specific reactions were blocked with a 2% BSA (Sigma Aldrich, A1470)/phosphate buffered saline solution. An anti-CEACAM5 antibody (R & D SYSTEMS, MAB41281, concentration: 1/100) was reacted with the specimen at 4° C. overnight, and thereafter, immunostaining signals were visualized using Histostar (trademark) (MBL, #8460) and DAB Substrate Solution (MBL, #8469). Finally, nuclear staining was carried out using hematoxylin (Sakura Finetek Japan Co., Ltd., #8650). The results we shown in FIG. 12.

From FIG. 11 and FIG. 12, it was confirmed that a complex of CEA-V2122 and Compound 1 is administered to a xenograft mouse model and the mouse model is then irradiated with alight with a wavelength of 690 nm, so that necrosis can be induced to the tumor of the xenograft mouse model.

### Example 6: Expression and Purification of HER2-V2122 Protein

V2122 is a mutant streptavidin described in Example 3 of International Publication WO2015/125820 (SEQ ID NO:4 shown in International Publication WO2015/125820). The amino acid sequence of V2122 is as set forth in SEQ ID NO: 1 in the sequence listing.

scFv-V2122 is prepared by binding a single-chain antibody (scFv) against HER2 (ERBB2) with the above-described V2122. This scFv-type anti-HER2 antibody is an scFv sequence described in Zhang H, et al, Therapeutic potential of an anti-HER2 single chain antibody-DM1 conjugates for the treatment of HER2-positive cancer. Signal Transduct Target Ther. 2017 May 19; 2: 17015. doi: 10.1038/sigtrans. 2017.15. The amino acid sequence of the scFv-type anti-HER2 antibody is as set forth in SEQ ID NO: 7 in the sequence listing. In addition, the structural drawing of HER2-V2122 prepared by binding the scFv-type anti-HER2 antibody with V2122 via an amino acid linker (GGGGGSGGGGG) (SEQ ID NO: 18) is shown in FIG. 13, and the amino acid sequence of HER2-V2122 is as set forth in SEQ ID NO:8 in the sequence listing.

For the expression of a HER2-V2122 fusion protein, the DNA codon of a HER2-V2122 gene sequence, in which a pelB signal for secretion and expression in Escherichia coli had been incorporated into the N-terminus and a 6×His-Tag sequence had been incorporated into the C-terminus, was optimized for Escherichia coli, thereby synthesizing an artificial gene. This amino acid sequence is as set forth in SEQ ID NO: 9 in the sequence listing, and the DNA sequence is as et forth in SEQ ID NO: 10 in the sequence listing.

As a specific protein expression vector, a vector prepared by incorporating a chaperone skp gene into MCS2 of a pETDuet1 vector was used. Regarding the skp gene, the DNA codon was optimized for Escherichia coli based on the amino acid sequence as set forth in SEQ ID NO: 6 in the sequence listing, thereby synthesizing an artificial gene. The synthesized skp gene was amplified by PCR, using the primers (AAGGAGATATACATATGGATAAAATTGCCATTGTTAATAT (SEQ ID NO: 12), and TTGAGATCTGCCATATGTTATTTCACTTGTTTCAGAACG (SEQ ID NO: 13)), and the amplified gene was then cloned into MCS2 of the pETDuel vector linearized with the restriction enzyme NdeI, using In-Fusion HD Cloning Kit, so as to obtain pETDuet_skp. Subsequently, the HER2-V2122 gene was incorporated into MCS1 of pETDuet_skp. Specifically, the artificially synthesized HER2-V2122 gene was amplified by PCR, using the primers (AGAAGGAGATATACCATGAAATATCTGCTGCCGAC (SEQ ID NO: 14), and CGCCGAGCTCGAATTTTAATGATGGTGATGATGATG (SEQ ID NO: 15)). Moreover, pETDuet_skp was linearized by PCR, using the primers (GGTATATCTCCTCTTAAAGTTAAAC (SEQ ID NO: 16), and AATTCGAGCTCGGCGCGCCTGCAG (SEQ ID NO: 17)). The CEA-V2122 amplified by PCR and the linearized pETDuet_skp were subjected to cloning using In-Fusion HD Cloning Kit. The cloned vector was confirmed by sequencing, in terms of the gene sequence incorporated therein, and thereafter, it was referred to as pETDuet_HER2-V2122_skp.

For the expression of the protein, pETDuet_HER2-V2122 skp was transformed into BL21(DE3) (Nippon Gen Co., Ltd) which was then pre-cultured in 2×YT medium (SIGMA-ALDRICH) at 37° C. overnight. The medium used in the pre-culture was added to a new medium for 100-fold dilution, and culture was then carried out at 37° C., until OD (600 nm) became 0.5 to 2.0. Subsequently, IPTG was added to the culture to a final concentration of 0.5 mM, and the obtained mixture was then cultured at 37° C. for 4 hours. Thereafter, a culture supernatant was recovered and was then preserved at 4′C.

The HER2-V2122 protein was roughly purified according to a batch method utilizing a 6×His-Tag added to the C-terminus. Specifically, cOmplete His-Ig Purification Resin equilibrated with buffer A (50 mM Tis-HCl, 0.2 M NaCl, 1 mM EDTA, and 5 mM Imidazole; pH 8.0) was added to the culture supernatant preserved at 4° C. The obtained mixture was stirred for 2 hours to overnight at 4° C., so that the protein was allowed to bind to the resin. Subsequently, the resin was recovered into a column, and a 20 column volume of washing operation was performed with buffer A. Thereafter, a roughly purified product of HER2-V2122 was recovered by elution with buffer B (50 mM Tris-HC, 0.2 M NaCl, 1 mM EDTA, and 400 mM Imidazole; pH 8.0).

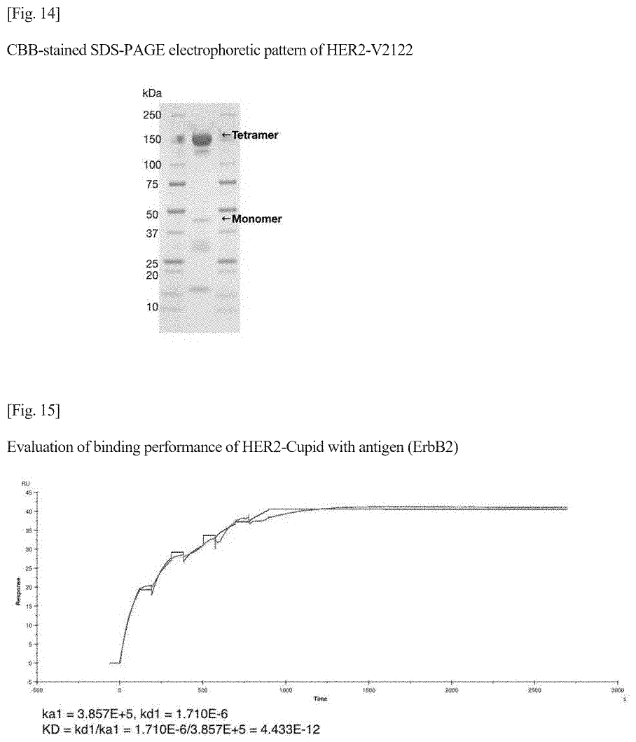

Subsequently, the roughly purified product was purified using a Protein L column. Specifically, 1 mL of Capto L (GE Healthcare) was filled into a PD-10 column and was then equilibrated with 10 column volume of PBS, and the aforementioned roughly purified product was then applied thereto. Thereafter the resultant was washed with 10 column volume of PBS, was then eluted with 10 mM glycine hydrochloride (pH 2.0), and was then subjected to centrifugal concentration using Vivaspin Turbo 15 (MWCO 100,000). Moreover, using PD-10 (GE Healthcare), the buffer was replaced with PBS, and centrifugal concentration was further carried out using Vivaspin Turbo 4 (MWCO 100,000) to obtain a finally purified product. The purified product was subjected to CBB staining and the purity of tetramer HER2-V2122 was assayed. The results are shown in FIG. 14. As an SDS-PAGE gel, Mini-PROTEAN TGX 4-15% (Bio-Rad) was used, and as a CBB staining solution, Bullet CBB Stain One (Ready To Use)(Nacalai Tesque, Inc.) was used.

From FIG. 14, it was confirmed that the purified HER2-V2122 comprises an approximately 150 kDa tetramer as a main component.

### Example 7: Evaluation of Performance of HER2-V2122 by SPR

The affinity of HER2-V2122 for the antigen CEACAM5 was evaluated using a surface plasmon resonance (SPR) measuring device, Biacore T200 (GE Healthcare Life Sciences). Specifically, Recombinant Hunan ErbB2/Fc Chimera, Carrier Free (R & D SYSTEMS, 1129-ER-050) was immobilized on Sensor Chip CM5 (GE Healthcare Life Sciences) using an amine-coupling kit (GE Healthcare Life Sciences). The final amount of the ligand immobilized was 279 RU. Moreover, with regard to the purified HER2-V2122, two-fold serial dilutions from 1E-08 M to 6.25E-10 M were prepared as analytes. Regarding interaction analysis, data were obtained by single-cycle kinetics analysis. Using Biacore T200 Evaluation Software, version 2.0, the obtained data were subjected to curve fitting in a bivalent analysis mode, and the following values were obtained: ka1=3.857E+5, and kd1=1.710E−6. Moreover since evaluation can be carried out at K_{D}=kd1/ka1 in the bivalent analysis, the evaluation value K_{D}=kd1/ka1=1.710E−6/3.857E+5=4.433E-12 was obtained. These results are shown in FIG. 15.

From the sensorgram and the calculated K_{D} value shown in FIG. 15, it was confirmed that HER-V2122 recognizes ErbB2 and strongly binds thereto.

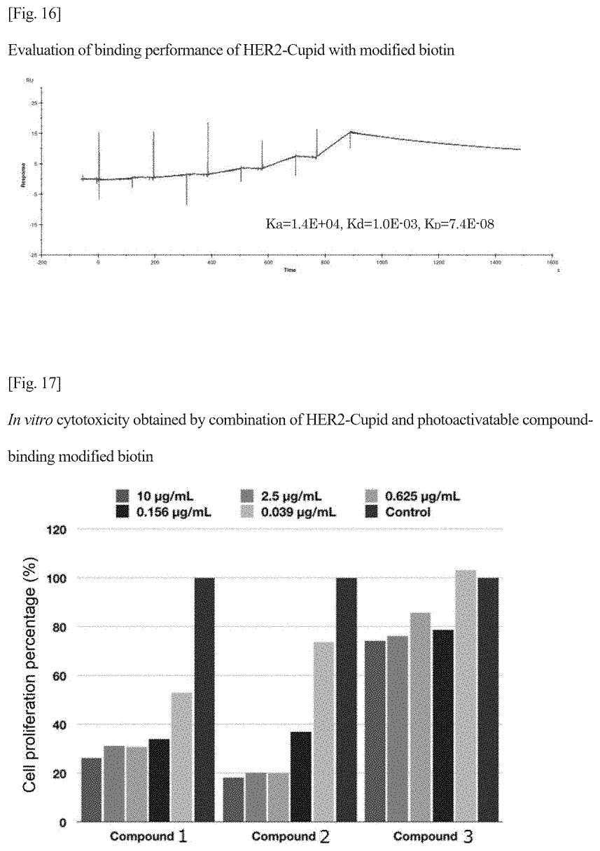

Furthermore, the interaction between HER2-V2122 and a modified biotin (Production Example 1, the compound represented by Formula 11) was also analyzed using Biacore T200. Specifically, an amine-coupling kit was used, a target value was set to be 5000 RU, and the purified CEA-V2122 was immobilized on Sensor Chip CM5. With regard to the concentrations of the analytes, 5 types of two-fold serial dilutions from 1E-08 M to 6.25E-10 M were used. Regarding interaction analysis, data were obtained by single-cycle kinetics analysis. Using Biacore 1200 Evaluation Software, version 2.0, the obtained data were subjected to curve fitting in a bivalent analysis mode, and the evaluation value of affinity was obtained. These results are shown in FIG. 16.

### Example 8: In Vitro Cytotoxicity Teat Using HER2-V2122 and Photoactivatable Compound-Labeled Modified Biotins

A cytotoxicity test was carried out using the photoactivatable compound-labeled modified biotins, namely, Compound 1, Compound 2, and Compound 3. These compounds are described in Japanese Patent Application No. 2018-149295. Compound 1, Compound 2, and Compound 3 am shown in FIG. 7. Specifically, SK-BR-3 cells cultured in a McCoy's medium supplemented with 10% FBS were seeded on a 96-well plate for cell culture, so that the cell count became 5×10³ cells/well and the amount of the culture solution became 50 μL/well, and thereafter, the cells were cultured overnight. A solution containing a complex of CEA-V2122 and a photoactivatable compound-labeled modified biotin was prepared, so that the molar ratio of CEA-V2122 and each compound became 1:2, and the solution was then incubated at room temperature for 10 minutes. Thereafter, the concentration of the reaction solution was adjusted with a culture solution, so that the final concentration of HER2-V2122 became 10 μg/mL. Regarding serial dilutions, 20 μg/mL was set to be an initial concentration, from 4-fold serial dilutions (5.0 μg/mL, 1.25 μg/mL, 0.312 μg/mL, and 0.078 μg/mL), 5 complex serial dilution solutions were prepared. Besides, a medium alone that contained no complex was used as a zero control

Such complex serial dilution solutions were each added in an amount of 50 μL/well to the cells cultured overnight, so that the final concentrations became 10 μg/mL, 2.5 μg/mL, 0.625 μg/mL, 0.156 μg/mL, and 0.039 μg/mL. One hour and two hours after addition of the complex, the cells wee irradiated with a light, using LED emitting a light having a wavelength of 690±10 nm, so that the irradiation energy became 100 J/cm². Thereafter, the cells were cultured for 48 hours, and thereafter, a comparison was made in terms of the number of surviving cells, using Cell Counting Kit-8 (DOJINDO LABORATORIES). Each condition was set to be n=3. The dosage and administration were determined in accordance with the instruction manuals included with the kit, and addition of the reagent, the mixture was incubated for 1.5 hours at 37° C., in a CO₂ incubator. Thereafter the absorbance at 450 nm was measured, and the mean value was then calculated, followed by background collection. The control was set to be 100%, and the ratio of cell proliferation to the control under each condition was calculated. The results are shown in FIG. 17.

As shown in FIG. 17, it was confirmed that the complex of HER2-Cupid and a photoactivatable compound-labeled modified biotin exhibits cytotoxicity in a concentration-dependent manner.

SEQ ID NO: 1
AEAGITGTWSDQLGDTFIVTAGADGALTGTYENAVGGAESRYVLTGRYDS
APATDGSGTALGWTVAWKNNSKNAHSATTWSGQYVGGADAKINTQWLLTS
GTTNANAWKSTLVGHDTFTKVKPSAASHHHHHH
(sm3E-scFv sequence)
SEQ ID NO: 2
QVKLEQSGAEVVKPGASVKLSCKASGFNIKDSYMHWLRQGPGQRLEWIGW
IDPENGDTEYAPKFQGKATFTTDTSANTAYLGLSSLRPEDTAVYYCNEGT
PTGPYYFDYWGQGTLVTVSSGGGGSGGGGSGGGGSENVLTQSPSSMSVSV
GDRVNIACSASSSVPYMHWLQQKPGKSPKLLIYLTSNLASGVPSRFSGSG
SGTDYSLTISSVQPEDAATYYCQQRSSYPLTFGGGTKLEIK
SEQ ID NO: 3
QVKLEQSGAEVVKPGASVKLSCKASGFNIKDSYMHWLRQGPGQRLEWIGW
IDPENGDTEYAPKFQGKATFTTDTSANTAYLGLSSLRPEDTAVYYCNEGT
PTGPYYFDYWGQGTLVTVSSGGGGSGGGGSGGGGSENVLTQSPSSMSVSV
GDRVNIACSASSSVPYMHWLQQKPGKSPKLLIYLTSNLASGVPSRFSGSG
SGTDYSLTISSVQPEDAATYYCQQRSSYPLTFGGGTKLEIKGGGGSGGGG
AEAGITGTWSDQLGDTFIVTAGADGALTGTYENAVGGAESRYVLTGRYDS
PATDGSGTALGWTVAWKNNSKNAHSATTWSGQYVGGADAKINTQWLLTSG
TTNANAWKSTLVGHDTFTKVKPSAASHHHHH
SEQ ID NO: 4
MKYLLPTAAAGLLLLAAQPAMAQVKLEQSGAEVVKPGASVKLSCKASGFN
IKDSYMHWLRQGPGQRLEWIGWIDPENGDTEYAPKFQGKATFTTDTSANT
AYLGLSSLRPEDTAVYYCNEGTPTGPYYFDYWGQGTLVTVSSGGGGSGGG
GSGGGGSENVLTQSPSSMSVSVGDRVNIACSASSSVPYMHWLQQKPGKSP
KLLIYLTSNLASGVPSRFSGSGSGTDYSLTISSVQPEDAATYYCQQRSSY
PLTFGGGTKLEIKGGGGSGGGGAEAGITGTWSDQLGDTFIVTAGADGALT
GTYENAVGGAESRYVLTGRYDSAPATDGSGTALGWTVAWKNNSKNAHSAT
TWSGQYVGGADAKINTQWLLTSGTTNANAWKSTLVGHDTFTKVKPSAASH
HHHHH
SEQ ID NO: 5
ATGAAATATCTGCTGCCGACCGCAGCAGCGGGTCTGCTGCTGCTGGCAGC
ACAGCCTGCAATGGCACAGGTTAAACTGGAACAGAGCGGTGCCGAAGTTG
TTAAACCGGGTGCAAGCGTTAAACTGAGCTGTAAAGCAAGCGGCTTAACA
TCAAAGATAGCTATATGCATTGGCTGCGTCAGGGTCCCGGGTCAGCGTCT
GGAATGGATTGGTTGGATTGATCCGGAAAATGGTGATACCGAATATGCAC
CGAAATTTCAGGGTAAAGCAACCTTTACCACCGATACCAGCGCAAATACC
GCATATCTGGGTCTGAGCAGCCTGCGTCCGGAAGATACCGCAGTGTATAT
TGTAATGAAGGCACCCCGACCGGTCCGTATTATTTCGATTATTGGGGTCA
GGGCACCCTGGTTACCGTTAGCAGCGGTGGTGGTGGTAGTGGTGGCGGTG
GTTCAGGCGGTGGCGGTAGCGAAAATGTTCTGACCCAGAGCCCGAGCACT
CATGAGCGTTAGCGTTGGTGATCGTGTTAATATTGCATGTAGCGCAAGCA
GCAGCGTTCCGTACATGCACTGGCTGCAGCAGAAACCGGGTAAAAGCCCG
AAACTGCTGATTFATCTGACCAGCAATCTGGCAAGCGGTGTTCCGAGCCG
TTTTAGCGGTAGCGGTAGTGGCACCGATTATAGCCTGACCATTAGCAGCG
TGCAGCCTGAAGATGCAGCAACCTATTATTGTCAGCAGCGTAGCAGTTAT
CCGCTGACCTTTGGTGGTGGCACCAAACTGGAAATTAAAGGGGGTGGTGG
CTCAGGTGGCGGAGGTGCAGAAGCAGGTATTACCGGTACATGGTCAGATC
AGCTGGGTGATACCTTTATTGTTACCGCAGGCGCAGATGGTGCACTGACC
GGCACCTATGAAAATGCAGTTGGTGGTGCAGAAAGCCGTTATGTGCTGAC
CGGTCGTTATGATAGCGCACCGGCAACCGATGGTAGCGGCACCGCACTGG
GTTGGACCGTTGCATGGAAAAATAACAGCAAAAATGCACATAGCGCAACC
ACCTGGTCAGGTCAGTATGTGGGTGGTGCCGATGCCAAAATTAACACCCA
GTGGCTGCTGACCAGCGGTACAACCAATGCAAATGCCTGGAAAAGTACCC
TGGTTGGTCATGATACATTCACCAAAGTTAAACCGAGCGCAGCAAGCCAT
CATCATCACCATCATTAA
SEQ ID NO: 6
MDKIAIVNMGSLFQQVAQKTGVSNTLENEFKGRASELQRMETDLQAKMKK
LQSMKAGSDRTKLEKDVMAQRQTFAQKAQAFEQDRARRSNEERGKLVTRI
QTAVKSVANSQDIDLVVDANAVAYNSSDVKDITADVLKQVK
SEQ ID NO: 7
EVQLVESGGGLVQPGGSLRLSCAASGFNIKDTYIHWVRQAPGKGLEWVAR
IYPTNGYTRYADSVKGRFTISADTSKNTAYLQMNSLRAEDTAVYYCSRWG
GDGFYAMDYWGQGTLVTVSSGGGGSGGGGSGGGGSGGGGSDIQMTQSPSS
LSASVGDRVTITCRASQDVNTAVAWYQQKPGKAPKLLIYSASFLYSGVPS
RFSGSRSGTDFTLTISSLQPEDFATYYCQQHYTTPPTFGQGTKVEIK
SEQ ID NO: 8
EVQLVESGGGLVQPGGSLRLSCAASGFNTKDTYHWVRQAPGKGLEWVARI
YPTNGYTRYADSVKGRFTISADTSKNTAYLQMNSLRAEDTAVYYCSRWGG
DGFYAMDYWGQGTLVTVSSGGGGSGGGGSGGGGSGGGGSDIQMTQSPSSL
SASVGDRVTITCRASQDVNTAVAWYQQKPGKAPKLLIYSASFLYSGVPSR
FSGSRSGTDFTLTISSLQPEDFATYYCQQHYTTPPTFGQGTKVEIKGGGG
GSGGGGGAEAGITGTWSDQLGDTFIVTAGADGALTGTYENAVGGAESRYV
LTGRYDSAPATDGSGTALGWTVAWKNNSKNAHSATTWSGQYVGGADAKIN
TQWLLTSGTTNANAWKSTLVGHDTFTKVKPSAASHHHHHH
SEQ ID NO: 9
MKYLLPTAAAGLLLLAAQPAMAEVQLVESGGGLVQPGGSLRLSCAASGFN
IKDTYIHWVRQAPGKGLEWVARIYPTNGYTRYADSVKGRFTISADTSKNT
AYLQMNSLRAEDTAVYYCSRWGGDGFYAMDYWGQGTLVTVSSGGGGSGGG
GSGGGGSGGGGSDIQMTQSPSSLSASVGDRVTITCRASQDVNTAVAWYQQ
KPGKAPKLLIYSASFLYSGVPSRFSGSRSGTDFTLTISSLQPEDFATYYC
QQHYTTPPTFGQGTKVEIKGGGGGSGGGGGAEAGITGTWSDQLGDTFIVT
AGADGALTGTYENAVGGAESRYVLTGRYDSAPATDGSGTALGWTVAWKNN
SKNAHSATTWSGQYVGGADAKINTQWLLTSGTTNANAWKSTLVGHDTFTK
VKPSAASHHHHHH
SEQ ID NO: 10
ATGAAATATCTGCTGCCGACCGCACTCAGCGGGTCTGCTGCTGCTGGCAC
TCACAGCCTGCAATGCTCAGAAGITCAGCTGGTTGAAACTCCTGTGGTGG
TCTGGTTCACTCCTGGTGGTAGCCTGCGTCTGAGCTGTGCAGCAAGCGGT
TTFYAACATTAAAGATACCTATATTCATTGGTGCGTCAGGCACCTGGTAA
AGGTCTGGAATGGGTTGCACGTATTTATCCGACCAATGGTTATACCCGTT
ATGCCGATAGCGTTAAAGGTCGTTTTACCATTAGCGCAGATACCAGCAAA
AATACCGCATACCTGCAGATGAATAGTCTGCGTGCAGAGGATACCGCAGT
GTATTATTGTAGCCGTTGGGGTGGTGATCTGTTTTTATGCAATGGATTAT
TGGGGTCAGGGCACCCTGGTTACCGTTAGCTCAGGTCTGAGGCCTGTTCC
GGTCTGCCTGACTGTTCCGGTGGAGGTGGCTCCGGTGGCGGAGGTTCCGA
TATTCAGATGACCCAGAGTCCGAGCAGCCTGAGCGCAAGCGTTGGTGATC
GTGTGACCATTACCTGTCGTGCAAGCCAGGATGTTAATACAGCAGTTGCA
TGGTATCAGCAGAAACCCGGGTAAAGCACCGAAACTGCTGATTTATACTC
GCAAGCTTTCTGTATAGCGGTGTTCCGAGCCGTTTTAGCGGTAGCCGTAG
CGGCACCGATTTTACCCTGACCATTAGCAGCCTGCAGCCGGAAGATTTTG
CAACCTATTATTGTCAGCAGCATFACACCACACCGCCTACCTTTGGCCAG
GGCACCAAAGITGAAATTAAAGGAGGTGGCGGTGGATCCGGCGGAGGTGG
CGGAGCAGAAGCAGGTATTACCGGTACATGGTCAGATCAGCTGGGTGATA
CCTTTATTGTTACCGCAGGCGCAGATGGTGCACTGACCGGCACCTATGAA
AATGCAGTTGGTGGTGCAGAAAGCCGTTATGTGCTGACCGGTCGTTATGA
TAGCGCACCGGCAACCGATGGTAGCGGCACCGCACTGGGTTGGACCGTTG
CATGGAAAAATAACAGCAAAAATGCACATAGCGCAACCACCTGGTCAGGT
CAGTATGTGGTGTGGTGCCGATGCCAAAATTAACACCCAGTGGCTGCTGA
CCAGCGGTACAACCAATGCAAATGCCTGGAAAAGTACCCTGGTTGGTCAT
GATACATTCACCAAAGTTAAACCGAGCGCAGCAAGCCATCATCATCACCA
TCATTAA

1. A fusion protein having the amino acid sequence as set forth in SEQ ID NO: 2 or SEQ ID NO: 7, a linker sequence, and the amino acid sequence as set forth in SEQ ID NO: 1 (provided that the amino acid sequence portion consisting of 6 histidine residues at the C-terminus thereof may be partially or entirely deleted), from the N-terminal side to the C-terminal side, in this order.

2. The fusion protein according to claim 1, wherein the number of amino acids of the linker sequence is 5 to 15.

3. The fusion protein according to claim 1, wherein the linker sequence consists of 4 to 14 glycine residues and 1 cysteine residue.

4. The fusion protein according to claim 1, wherein the linker sequence is Gly-Gly-Gly-Gly-Ser-Gly-Gly-Gly-Gly or Gly-Gly-Gly-Gly-Gly-Ser-Gly-Gly-Gly-Gly-Gly.

5. A fusion protein having the amino acid sequence as set forth in SEQ ID NO: 3 or SEQ ID NO: 8 (provided that the amino acid sequence portion consisting of 6 histidine residues at the C-terminus thereof may be partially or entirely deleted).

6. The fusion protein according to claim 1, further having a secretory signal sequence.

7. A fusion protein having the amino acid sequence as set forth in SEQ ID NO: 4 or SEQ ID NO: 9 (provided that the amino acid sequence portion consisting of 6 histidine residues at the C-terminus thereof may be partially or entirely deleted).

8. A nucleic acid encoding a fusion protein having the amino acid sequence as set forth in SEQ ID NO: 3 or SEQ ID NO: 8 (provided that the amino acid sequence portion consisting of 6 histidine residues at the C-terminus thereof may be partially or entirely deleted).

9. A nucleic acid encoding a fusion protein having the amino acid sequence as set forth in SEQ ID NO: 4 or SEQ ID NO: 9 (provided that the amino acid sequence portion consisting of 6 histidine residues at the C-terminus thereof may be partially or entirely deleted).

10. A cancer therapeutic agent or a cancer diagnostic agent, comprising the fusion protein according to claim 1.

11. A kit for treating or diagnosing cancer, comprising (1) the fusion protein according to claim 1, and (2) a conjugate of a compound represented by the following formula (1) or a salt thereof and a diagnostic substance or a therapeutic substance: 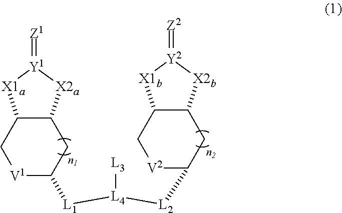
wherein
X1a, X1b, X2a and X2b each independently represent O or NH,
Y¹ and Y² each independently represent C or S,
Z¹ and Z² each independently represent O, S or NH,
V¹ and V² each independently represent S or S⁺—O⁻,
n1 and n2 each independently represent an integer of 0 or 1,
L₁ and L₂ each independently represent a divalent linking group,
L₃ represents a group comprising a functional group capable of binding to the diagnostic substance or the therapeutic substance at the terminus, and
L₄ represents a trivalent linking group.

12. The kit according to claim 11, wherein the diagnostic substance or the therapeutic substance is a phthalocyanine dye.


SEQUENCE LISTING (18 sequences)

SEQ ID NO: 1 - protein, 133 aa, Artificial Sequence
  Description of Artificial Sequence Recombinant protein
AEAGITGTWSDQLGDTFIVTAGADGALTGTYENAVGGAESRYVLTGRYDSAPATDGSGTALGWTVAWKNNSKNAHSATTWSGQYVGGADAKINTQWLLTSGTTNANAWKSTLVGHDTFTKVKPSAASHHHHHH

SEQ ID NO: 2 - protein, 241 aa, Artificial Sequence
  Description of Artificial Sequence Recombinant protein
QVKLEQSGAEVVKPGASVKLSCKASGFNIKDSYMHWLRQGPGQRLEWIGWIDPENGDTEYAPKFQGKATFTTDTSANTAYLGLSSLRPEDTAVYYCNEGTPTGPYYFDYWGQGTLVTVSSGGGGSGGGGSGGGGSENVLTQSPSSMSVSVGDRVNIACSASSSVPYMHWLQQKPGKSPKLLIYLTSNLASGVPSRFSGSGSGTDYSLTISSVQPEDAATYYCQQRSSYPLTFGGGTKLEIK

SEQ ID NO: 3 - protein, 383 aa, Artificial Sequence
  Description of Artificial Sequence Recombinant protein
QVKLEQSGAEVVKPGASVKLSCKASGFNIKDSYMHWLRQGPGQRLEWIGWIDPENGDTEYAPKFQGKATFTTDTSANTAYLGLSSLRPEDTAVYYCNEGTPTGPYYFDYWGQGTLVTVSSGGGGSGGGGSGGGGSENVLTQSPSSMSVSVGDRVNIACSASSSVPYMHWLQQKPGKSPKLLIYLTSNLASGVPSRFSGSGSGTDYSLTISSVQPEDAATYYCQQRSSYPLTFGGGTKLEIKGGGGSGGGGAEAGITGTWSDQLGDTFIVTAGADGALTGTYENAVGGAESRYVLTGRYDSAPATDGSGTALGWTVAWKNNSKNAHSATTWSGQYVGGADAKINTQWLLTSGTTNANAWKSTLVGHDTFTKVKPSAASHHHHHH

SEQ ID NO: 4 - protein, 405 aa, Artificial Sequence
  Description of Artificial Sequence Recombinant protein
MKYLLPTAAAGLLLLAAQPAMAQVKLEQSGAEVVKPGASVKLSCKASGFNIKDSYMHWLRQGPGQRLEWIGWIDPENGDTEYAPKFQGKATFTTDTSANTAYLGLSSLRPEDTAVYYCNEGTPTGPYYFDYWGQGTLVTVSSGGGGSGGGGSGGGGSENVLTQSPSSMSVSVGDRVNIACSASSSVPYMHWLQQKPGKSPKLLIYLTSNLASGVPSRFSGSGSGTDYSLTISSVQPEDAATYYCQQRSSYPLTFGGGTKLEIKGGGGSGGGGAEAGITGTWSDQLGDTFIVTAGADGALTGTYENAVGGAESRYVLTGRYDSAPATDGSGTALGWTVAWKNNSKNAHSATTWSGQYVGGADAKINTQWLLTSGTTNANAWKSTLVGHDTFTKVKPSAASHHHHHH

SEQ ID NO: 5 - DNA, 1218 bp, Artificial Sequence
  Description of Artificial Sequence Recombinant DNA
atgaaatatctgctgccgaccgcagcagcgggtctgctgctgctggcagcacagcctgcaatggcacaggttaaactggaacagagcggtgccgaagttgttaaaccgggtgcaagcgttaaactgagctgtaaagcaagcggctttaacatcaaagatagctatatgcattggctgcgtcagggtccgggtcagcgtctggaatggattggttggattgatccggaaaatggtgataccgaatatgcaccgaaatttcagggtaaagcaacctttaccaccgataccagcgcaaataccgcatatctgggtctgagcagcctgcgtccggaagataccgcagtgtattattgtaatgaaggcaccccgaccggtccgtattatttcgattattggggtcagggcaccctggttaccgttagcagcggtggtggtggtagtggtggcggtggttcaggcggtggcggtagcgaaaatgttctgacccagagcccgagcagcatgagcgttagcgttggtgatcgtgttaatattgcatgtagcgcaagcagcagcgttccgtacatgcactggctgcagcagaaaccgggtaaaagcccgaaactgctgatttatctgaccagcaatctggcaagcggtgttccgagccgttttagcggtagcggtagtggcaccgattatagcctgaccattagcagcgtgcagcctgaagatgcagcaacctattattgtcagcagcgtagcagttatccgctgacctttggtggtggcaccaaactggaaattaaagggggtggtggctcaggtggcggaggtgcagaagcaggtattaccggtacatggtcagatcagctgggtgatacctttattgttaccgcaggcgcagatggtgcactgaccggcacctatgaaaatgcagttggtggtgcagaaagccgttatgtgctgaccggtcgttatgatagcgcaccggcaaccgatggtagcggcaccgcactgggttggaccgttgcatggaaaaataacagcaaaaatgcacatagcgcaaccacctggtcaggtcagtatgtgggtggtgccgatgccaaaattaacacccagtggctgctgaccagcggtacaaccaatgcaaatgcctggaaaagtaccctggttggtcatgatacattcaccaaagttaaaccgagcgcagcaagccatcatcatcaccatcattaa

SEQ ID NO: 6 - protein, 141 aa, Artificial Sequence
  Description of Artificial Sequence Recombinant protein
MDKIAIVNMGSLFQQVAQKTGVSNTLENEFKGRASELQRMETDLQAKMKKLQSMKAGSDRTKLEKDVMAQRQTFAQKAQAFEQDRARRSNEERGKLVTRIQTAVKSVANSQDIDLVVDANAVAYNSSDVKDITADVLKQVK

SEQ ID NO: 7 - protein, 247 aa, Artificial Sequence
  Description of Artificial Sequence Recombinant protein
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSGGGGSGGGGSGGGGSGGGGSDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIK

SEQ ID NO: 8 - protein, 391 aa, Artificial Sequence
  Description of Artificial Sequence Recombinant protein
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSGGGGSGGGGSGGGGSGGGGSDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKGGGGGSGGGGGAEAGITGTWSDQLGDTFIVTAGADGALTGTYENAVGGAESRYVLTGRYDSAPATDGSGTALGWTVAWKNNSKNAHSATTWSGQYVGGADAKINTQWLLTSGTTNANAWKSTLVGHDTFTKVKPSAASHHHHHH

SEQ ID NO: 9 - protein, 413 aa, Artificial Sequence
  Description of Artificial Sequence Recombinant protein
MKYLLPTAAAGLLLLAAQPAMAEVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSGGGGSGGGGSGGGGSGGGGSDIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKGGGGGSGGGGGAEAGITGTWSDQLGDTFIVTAGADGALTGTYENAVGGAESRYVLTGRYDSAPATDGSGTALGWTVAWKNNSKNAHSATTWSGQYVGGADAKINTQWLLTSGTTNANAWKSTLVGHDTFTKVKPSAASHHHHHH

SEQ ID NO: 10 - DNA, 1242 bp, Artificial Sequence
  Description of Artificial Sequence Recombinant DNA
atgaaatatctgctgccgaccgcagcagcgggtctgctgctgctggcagcacagcctgcaatggcagaagttcagctggttgaaagcggtggtggtctggttcagcctggtggtagcctgcgtctgagctgtgcagcaagcggttttaacattaaagatacctatattcattgggtgcgtcaggcacctggtaaaggtctggaatgggttgcacgtatttatccgaccaatggttatacccgttatgccgatagcgttaaaggtcgttttaccattagcgcagataccagcaaaaataccgcatacctgcagatgaatagtctgcgtgcagaggataccgcagtgtattattgtagccgttggggtggtgatggtttttatgcaatggattattggggtcagggcaccctggttaccgttagctcaggtggaggcggttccggtggcggaggttccggtggaggtggctccggtggcggaggttccgatattcagatgacccagagtccgagcagcctgagcgcaagcgttggtgatcgtgtgaccattacctgtcgtgcaagccaggatgttaatacagcagttgcatggtatcagcagaaaccgggtaaagcaccgaaactgctgatttatagcgcaagctttctgtatagcggtgttccgagccgttttagcggtagccgtagcggcaccgattttaccctgaccattagcagcctgcagccggaagattttgcaacctattattgtcagcagcattacaccacaccgcctacctttggccagggcaccaaagttgaaattaaaggaggtggcggtggatccggcggaggtggcggagcagaagcaggtattaccggtacatggtcagatcagctgggtgatacctttattgttaccgcaggcgcagatggtgcactgaccggcacctatgaaaatgcagttggtggtgcagaaagccgttatgtgctgaccggtcgttatgatagcgcaccggcaaccgatggtagcggcaccgcactgggttggaccgttgcatggaaaaataacagcaaaaatgcacatagcgcaaccacctggtcaggtcagtatgtgggtggtgccgatgccaaaattaacacccagtggctgctgaccagcggtacaaccaatgcaaatgcctggaaaagtaccctggttggtcatgatacattcaccaaagttaaaccgagcgcagcaagccatcatcatcaccatcattaa

SEQ ID NO: 11 - protein, 9 aa, Artificial Sequence
  Description of Artificial Sequence Recombinant linker
GGGGSGGGG

SEQ ID NO: 12 - DNA, 40 bp, Artificial Sequence
  Description of Artificial Sequence Synthesized DNA
aaggagatatacatatggataaaattgccattgttaatat

SEQ ID NO: 13 - DNA, 39 bp, Artificial Sequence
  Description of Artificial Sequence Synthesized DNA
ttgagatctgccatatgttatttcacttgtttcagaacg

SEQ ID NO: 14 - DNA, 35 bp, Artificial Sequence
  Description of Artificial Sequence Synthesized DNA
agaaggagatataccatgaaatatctgctgccgac

SEQ ID NO: 15 - DNA, 36 bp, Artificial Sequence
  Description of Artificial Sequence Synthesized DNA
cgccgagctcgaattttaatgatggtgatgatgatg

SEQ ID NO: 16 - DNA, 26 bp, Artificial Sequence
  Description of Artificial Sequence Synthesized DNA
ggtatatctccttcttaaagttaaac

SEQ ID NO: 17 - DNA, 24 bp, Artificial Sequence
  Description of Artificial Sequence Synthesized DNA
aattcgagctcggcgcgcctgcag

SEQ ID NO: 18 - protein, 11 aa, Artificial Sequence
  Description of Artificial Sequence Recombinant linker
GGGGGSGGGGG